(19) United States
(12) Patent Application Publication
(10) Pub. No.: US 2013/0303551 A1
(43) Pub. Date: Nov. 14, 2013

(54) PYRIMIDINONE DERIVATIVES AS FATTY ACID SYNTHASE INHIBITORS

(71) Applicants: Nicholas D. Adams, Collegeville, PA (US); Alexander Joseph Reif, Collegeville, PA (US); Kenneth Wiggall, Collegeville, PA (US)

(72) Inventors: Nicholas D. Adams, Collegeville, PA (US); Alexander Joseph Reif, Collegeville, PA (US); Kenneth Wiggall, Collegeville, PA (US)

(21) Appl. No.: 13/978,785

(22) PCT Filed: Jan. 10, 2012
(86) PCT No.: PCT/US12/20733

§ 371 Date: Jul. 9, 2013

Related U.S. Application Data
(60) Provisional application No. 61/431,160, filed on Jan. 10, 2011.

(51) Int. Cl. C07D 487/04; C07D 498/04; C07D 405/14; A61K 45/06; C07D 403/14; A61K 31/506; C07D 403/06; A61K 31/519; A61K 31/517

(52) U.S. Cl. CPC C07D 487/04; A61K 31/519; C07D 498/04; C07D 405/14; A61K 31/517; C07D 403/14; A61K 31/506; C07D 403/06; A61K 45/06
USPC 514/260.1; 544/262; 514/262.1; 544/255; 544/289; 514/266.24; 514/266.2; 544/319; 514/269

(57) ABSTRACT

This invention relates to the use of pyrimidinone derivatives for the modulation, notably the inhibition of the activity or function of fatty acid synthase (FAS). Suitably, the present invention relates to the use of pyrimidinones in the treatment of cancer.

### FIELD OF INVENTION

This invention relates to novel pyrimidinones which are inhibitors of fatty acid synthase (FAS), to pharmaceutical compositions containing them, to processes for their preparation, and to their use in therapy for the treatment of cancers.

### BACKGROUND

Fatty acids have an essential role in a variety of cellular processes including building blocks for membranes, anchors for targeting membrane proteins, precursors in the synthesis of lipid second messengers and as a medium to store energy, Menendez J S and Lupu R, Fatty acid synthase and the lipogenic phenotype in cancer pathogenesis, Nature Reviews Cancer, 7: 763-777 (2007). Fatty acids can either be obtained from the diet or can be synthesized de novo from carbohydrate precursors. The biosynthesis of the latter is catalyzed by the muliti-functional homodimeric FAS. FAS synthesizes long chain fatty acids by using acetyl-CoA as a primer and Malonyl Co-A as a 2 carbon donor, and NADPH as a reducing equivalents (Wakil S J, Lipids, Structure and function of animal fatty acid synthase, 39: 1045-1053 (2004), Asturias F J et al., Structure and molecular organization of mammalian fatty acid synthase, Nature Struct. Mol. Biol. 12:225-232 (2005), Maier T, et al., Architecture of Mammalian Fatty Acid Synthase at 4.5Å Resolution, Science 311:1258-1262 (2006).

De novo fatty acid synthesis is active during embryogenesis and in fetal lungs where fatty acids are used for the production of lung surfactant. In adults, most normal human tissues preferentially acquire fatty acids from the diet. Therefore, the level of de novo lipogensis and expression of liopogenic enzymes is low, Weiss L, et al., Fatty-acid biosynthesis in man, a pathway of minor importance. Purification, optimal assay conditions, and organ distribution of fatty-acid synthase. Biological Chemistry Hoppe-Seyler 367(9):905-912 (1986). In contrast, many tumors have high rates of de novo fatty acid synthesis Medes G, et al., Metabolism of Neoplastic Tissue. IV. A Study of Lipid Synthesis in Neoplastic Tissue Slices in Vitro, Can Res, 13:27-29, (1953). FAS has now been shown to be overexpressed in numerous cancer types including prostate, ovary, colon, endometrium lung, bladder, stomach and kidney Kuhajda F P, Fatty-acid synthase and human cancer: new perspectives on its role in tumor biology, Nutrition; 16:202-208 (2000). This differential expression and function of FAS in tumors and normal cells provide an approach for cancer therapy with the potential of a substantial therapeutic window.

Pharmacological and small interference RNA mediated inhibition of FAS has demonstrated a preferential inhibition of cancer cell proliferation. Additionally these inhibitors induce apoptosis in cancers cells in vitro and retard growth in human tumors in murine xenograft models in vivo, Menendez J S and Lupu R, Nature Reviews Cancer, 7: 763-777 (2007). Based upon these findings, FAS is considered a major potential target of antineoplastic intervention.

### SUMMARY OF THE INVENTION

This invention relates to compound of the Formula (I), as shown below

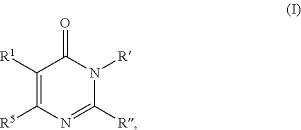

wherein, one of R′ and R″ is

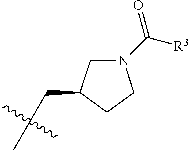

and the other of R′ and R″ is

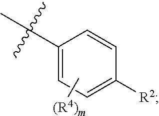

wherein
R¹ and R⁵ are each independently selected from the group consisting of: hydrogen, C₁-C₆alkyl, —C₁-C₆alkoxy, hydroxyl, halogen, —NR⁷R⁸, —C₁-C₆alkylNR⁷R⁸, cyano, C₄₋C₆heterocycloalkyl, —OC₁-C₄alkyl, and —C(O)NRₐR_{b},
   in which Rₐ and R_{b} are independently hydrogen, C₁-C₆alkyl, or C₃₋C₇cycloalkyl, or Rₐ and R_{b} taken together with the atoms to which they are connected form a C₄-C₆heterocycloalkyl;
R⁷ is selected from the group consisting of hydrogen, C₁-C₄alkyl, C₃-C₇cycloalkyl, —C₁-C₃alkylC₃-C₇cycloalkyl, phenyl, and —C₁-C₃alkylphenyl;
R⁸ is hydrogen, C₁-C₄alkyl, C₃-C₇cycloalkyl, or —C₁-C₃alkylC₃-C₇cycloalkyl;
   or R¹ and R⁵ taken together with the atoms to which they are connected form a 5- or 6-membered ring, which ring optionally contains one or two heteroatoms and is optionally substituted by 1 to 2 groups selected from: halogen, C₁-C₄alkoxy, and C₁-C₄alkyl;
R² is phenyl, 5- or 6-membered heteroaryl, naphthyl, or 9- or 10-membered heterocyclyl; wherein said phenyl, 5- or 6-membered heteroaryl, naphthyl, 9- or 10-membered heterocyclyl, is optionally substituted with 1 to 3 substituents independently selected from halogen, C₁-C₄alkyl, —CF₃,
   C₃-C₇cycloalkyl, —C(O)C₁-C₄alkyl,
   —C(O)C₃-C₇cycloalkyl, —CO(phenyl), —C₁-C₄(═O)OH,
   —C(═O)OC₁-C₄alkyl, —CONR⁷R⁸, phenyl, —SO₂C₁-C₄alkyl, —SO₂NR⁷R⁸, cyano, oxo, hydroxyl, C₁-C₄alkoxy, C₃-C₇cycloalkoxy, hydroxyC₁-C₄alkyl-,
   C₁-C₄alkoxyC₁-C₄alkyl-,—OCF₃, —NR⁷R⁸, R⁷R⁸NC₁-C₄alkyl-, —NR⁷C(O)C₁-C₄alkyl, —NR⁷CONR⁷R⁸, —NR⁷SO₂C₁-C₄alkyl, —NR⁷SO₂NR⁷R⁸, and R⁹;
R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁷R⁸;
R³ is selected from the group consisting of C₁-C₆alkyl, —CF₃, C₃-C₇cycloalkyl, C₁-C₄alkoxy, OC₁₋₆ alkyl, R⁷R⁸NC₁-C₄alkyl-, and —NR⁷R⁸; wherein said C₃-C₇cycloalkyl is optionally substituted 1 or 2 times independently by halogen or C₁-C₄alkyl;
each R⁴ is selected from the group consisting of: hydroxyl, C₁-C₆alkyl, C₁-C₆alkoxy and halogen;
m is 0 to 3; or a pharmaceutically acceptable salt thereof.

This invention also relates to pharmaceutical compositions, which comprise compounds of Formula (I) and pharmaceutically acceptable carriers.

This invention also relates to methods of treating cancer which comprises administering an effective amount of a compound of Formula (I) to a human in need thereof.

This invention also relates to methods of treating cancer which comprise co-administering a compound of Formula (I) and a second compound to a human in need thereof.

### DETAILED DESCRIPTION OF THE INVENTION

This invention also relates to compound of the Formula (I)(A), as shown below

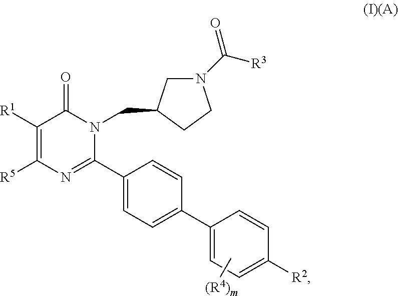

wherein
R¹ and R⁵ are each independently selected from the group consisting of: hydrogen, C₁-C₆alkyl, —C₁-C₆alkoxy, hydroxyl, halogen, —NR⁷R⁸, —C₁-C₆alkylNR⁷R⁸, cyano, C₄-C₆heterocycloalkyl, —OC₁-C₄alkyl, and —C(O)NRₐR_{b},
   in which Rₐ and R_{b} are independently hydrogen, C₁-C₆alkyl, or C₃-C₇cycloalkyl, or Rₐ and R_{b} taken together with the atoms to which they are connected form a C₄-C₆heterocycloalkyl;
R⁷ is selected from the group consisting of hydrogen, C₁-C₄alkyl, C₃-C₇cycloalkyl, —C₁-C₃alkylC₃-C₇cycloalkyl, phenyl, and —C₁-C₃alkylphenyl;
R⁸ is hydrogen, C₁-C₄alkyl, C₃-C₇cycloalkyl, or —C₁-C₃alkylC₃-C₇cycloalkyl;
   or R¹ and R⁵ taken together with the atoms to which they are connected form a 5- or 6-membered ring, in which the ring optionally contains one or two heteroatoms and is optionally substituted by 1 to 2 groups selected from: halogen, C₁-C₄alkoxy, and C₁-C₄alkyl;
R² is selected from the group consisting of: phenyl, 5- or 6-membered heteroaryl, naphthyl, or 9- or 10-membered heterocyclyl; wherein said phenyl, 5- or 6-membered heteroaryl, naphthyl, 9- or 10-membered heterocyclyl, is optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁷R⁸, phenyl, —SO₂C₁-C₄alkyl, —SO₂NR⁷R⁸, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁷R⁸,
   R⁷R⁸NC₁-C₄alkyl-, —NR⁷C(O)C₁-C₄alkyl, —NR⁷CONR⁷R⁸, —NR⁷SO₂C₁-C₄alkyl, —NR⁷SO₂NR⁷R⁸, and R⁹;
R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁷R⁸_{;}
R₃ is selected from the group consisting of C₁-C₆alkyl, —CF₃, C₃-C₇cycloalkyl, C₁-C₄alkoxy, OC₁-C₆alkyl, R⁷R⁸NC₁-C₄alkyl-, and —NR⁷R⁸; wherein said C₃-C₇cycloalkyl is optionally substituted 1 or 2 times independently by halogen or C₁-C₄alkyl;
each R⁴ is selected from the group consisting of: hydroxyl, C₁-C₆alkyl, alkoxy and halogen;
m is 0 to 3; or a pharmaceutically acceptable salt thereof.

This invention also relates to compounds of Formula (I)(B),

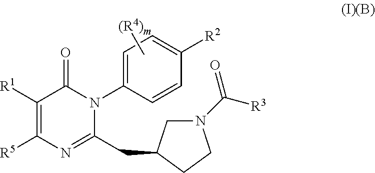

wherein
R¹ and R₅ are each independently selected from the group consisting of: hydrogen, C₁-C₆alkyl, —C₁-C₆alkoxy, hydroxyl, halogen, —NR⁷R⁸, —C₁₋₆alkylNR⁷R⁸, cyano, C₄-C₆heterocycloalkyl, —OC₁-C₄alkyl, and C(O)NRₐR_{b},
   in which Rₐ and R_{b} are independently hydrogen, C₁-C₆alkyl, or C₃-C₇cycloalkyl, or Rₐ and R_{b} taken together with the atoms to which they are connected form a C₄-C₆heterocycloalkyl;
R⁷ is selected from the group consisting of hydrogen, C₁-C₄alkyl, C₃-C₇cycloalkyl, —C₁-C₃alkylC₃-C₇cycloalkyl, phenyl, and C₁-C₃alkylphenyl;
R⁸ is hydrogen, C₁-C₄alkyl, C₃-C₇cycloalkyl, or C₁-C₃alkyl C₃-C₇cycloalkyl;
   or R₁ and R₅ taken together with the atoms to which they are connected form a 5- or 6-membered ring, which ring optionally contains one or two heteroatoms and is optionally substituted by 1 to 2 groups selected from: halogen, C₁-C₄alkoxy, and C₁-C₄alkyl;
R² is phenyl, 5- or 6-membered heteroaryl, naphthyl, or 9- or 10-membered heterocyclyl; wherein said phenyl, 5- or 6-membered heteroaryl, naphthyl, 9- or 10-membered heterocyclyl, is optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁷R⁸, phenyl, —SO₂C₁-C₄alkyl, —SO₂NR⁷R⁸, cyano, oxo, hydroxyl, C₁-C₄alkoxy, C₃-C₇cycloalkoxy, hydroxyC₁-C₄alkyl-,
   C₁-C₄alkoxyC₁-C₄alkyl-,-0CF₃, —NR⁷R⁸, R⁷R⁸NC₁-C₄alkyl-, —NR⁷C(O)C₁-C₄alkyl, —NR⁷CONR⁷R⁸, —NR⁷SO₂C₁-C₄alkyl, —NR⁷SO₂NR⁷R⁸, and R⁹;
R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and NR⁷R⁸;
R³ is selected from the group consisting of C₁-C₆alkyl, —CF₃, C₃-C₇cycloalkyl, C₁-C₄alkoxy, OC₁₋₆alkyl, R⁷R⁸NC₁-C₄alkyl-, and —NR⁷R⁸; wherein said C₃-C₇cycloalkyl is optionally substituted 1 or 2 times independently by halogen or C₁-C₄alkyl
each R₄ is selected from the group consisting of: hydroxyl, C₁-C₆alkyl,
C₁-C₆alkoxy and halogen;
m is 0 to 3; or a pharmaceutically acceptable salt thereof.

This invention also relates to compounds of Formula (I), (I)(A) or (I)(B), wherein R₃ is cyclopropyl.

This invention also relates to compounds of Formula (I), (I)(A) or (I)(B), wherein R¹ and R⁵ are each independently selected from the group consisting of: hydrogen, C₁-C₆alkyl, C₁-C₆alkoxy, hydroxyl, halogen, —NR⁷R⁸, cyano, heterocycloalkyl and C(O)NRₐR_{b}, in which Rₐ and R_{b} are hydrogen, C₁-C₆alkyl, C₃-C₇cycloalkyl.

This invention also relates to compounds of Formula (I), (I)(A) or (I)(B), wherein R¹ and R⁵ taken together with the atoms to which they are connected form a 5- or 6-membered ring, which ring optionally contains one or two heteroatoms atoms and is optionally substituted by 1 to 2 groups selected from: halogen, C₁-C₆alkoxy, and C₁-C₆alkyl.

This invention also relates to compounds of Formula (I), (I)(A) or (I)(B), wherein m is 0.

This invention also relates to compounds of Formula (I), (I)(A) or (I)(B), wherein m is 1

This invention also relates to compounds of Formula (I), (I)(A) or (I)(B), wherein R² is phenyl optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, —NHSO₂NR⁵R⁶, and R⁹.

This invention also relates to compounds of Formula (I), (I)(A) or (I)(B), wherein R² is selected from furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl, all of which are optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —C(O)phenyl, —C₁-C₄(═O)OH, —C(═O)OC₁-C₄alkyl,

—CO₂C₁-C₄alkyl, —C(O)NR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, and —NHSO₂NR⁵R⁶.

This invention also relates to compounds of Formula (I), (I)(A) or (I)(B), wherein R² is naphthyl optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, —NHSO₂NR⁵R⁶, and R⁹.

This invention also relates to compounds of Formula (I), (I)(A) or (I)(B),

wherein R² is selected from benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzothiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzothiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl, wherein said benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzothiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzothiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl, all of which are optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —C(O)phenyl, —C₁-C₄(═O)OH, —C(═O)OC₁-C₄alkyl, —C(O)NR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)NR⁵R⁶, —NR⁶SO₂C₁-C₄alkyl, —NR⁶SO₂NR⁵R⁶, and R⁹.

This invention also relates to compounds of Formula (I), (I)(A) or (I)(B), wherein R² is selected from phenyl and quinolinyl.

This invention also relates to the following compounds or pharmaceutically acceptable salt thereof:
5-{[(3R)-1-(cyclopropylcarbonyl)-3-pyrrolidinyl]methyl}-6-[4-(1H-indol-5-yl)phenyl]-1-methyl-1,5-dihydro-4H-pyrazolo[3,4-d]pyrimidin-4-one;
N′-[4′-(5-{[(3R)-1-(cyclopropylcarbonyl)-3-pyrrolidinyl]methyl}-1-methyl-4-oxo-4,5-dihydro-1H-pyrazolo[3,4-d]pyrimidin-6-yl)-3-biphenylyl]-N,N-dimethylsulfamide;
5-{[(3R)-1-(cyclopropylcarbonyl)-3-pyrrolidinyl]methyl}-6-[4-(1H-indol-6-yl)phenyl]-1-methyl-1,5-dihydro-4H-pyrazolo[3,4-d]pyrimidin-4-one;
6-[4-(1-benzofuran-5-yl)phenyl]-5-{[(3R)-1-(cyclopropylcarbonyl)-3-pyrrolidinyl]methyl}-1-methyl-1,5-dihydro-4H-pyrazolo[3,4-d]pyrimidin-4-one;
5-{[(3R)-1-(cyclopropylcarbonyl)-3-pyrrolidinyl]methyl}-6-[4-(1H-indazol-5-yl)phenyl]-1-methyl-1,5-dihydro-4H-pyrazolo[3,4-d]pyrimidin-4-one;
5-{[(3R)-1-(cyclopropylcarbonyl)-3-pyrrolidinyl]methyl}-1-methyl-6-[4-(6-quinolinyl)phenyl]-1,5-dihydro-4H-pyrazolo[3,4-d]pyrimidin-4-one;
5-{[(3R)-1-(cyclopropylcarbonyl)-3-pyrrolidinyl]methyl}-1-methyl-6-[4-(7-quinolinyl)phenyl]-1,5-dihydro-4H-pyrazolo[3,4-d]pyrimidin-4-one;
6-[4-(1,3-benzothiazol-5-yl)phenyl]-5-{[(3R)-1-(cyclopropylcarbonyl)-3-pyrrolidinyl]methyl}-1-methyl-1,5-dihydro-4H-pyrazolo[3,4-d]pyrimidin-4-one;
5-[4-(1-benzofuran-5-yl)phenyl]-6-{[(3R)-1-(cyclopropylcarbonyl)-3-pyrrolidinyl]methyl}-2-methyl[1,3]oxazolo[5,4-d]pyrimidin-7(6H)-one;
4′-(6-{[(3R)-1-(cyclopropylcarbonyl)-3-pyrrolidinyl]methyl}-2-methyl-7-oxo-6,7-dihydro [1,3]oxazolo[5,4-d]pyrimidin-5-yl)-4-biphenylcarbonitrile;
6-{[(3R)-1-(cyclopropylcarbonyl)-3-pyrrolidinyl]methyl}-5-[4-(1H-indazol-5-yl)phenyl]-2-methyl[1,3]oxazolo[5,4-d]pyrimidin-7(6H)-one;
5-[4-(1,3-benzothiazol-5-yl)phenyl]-6-{[(3R)-1-(cyclopropylcarbonyl)-3-pyrrolidinyl]methyl}-2-methyl[1,3]oxazolo[5,4-d]pyrimidin-7(6H)-one;
6-{[(3R)-1-(cyclopropylcarbonyl)-3-pyrrolidinyl]methyl}-5-[4-(1H-indol-5-yl)phenyl]-2-methyl[1,3]oxazolo[5,4-d]pyrimidin-7(6H)-one;
6-{[(3S)-1-(cyclopropylcarbonyl)-3-pyrrolidinyl]methyl}-5-[4-(1H-indol-6-yl)phenyl]-1-methyl-1,5-dihydro-4H-pyrazolo[3,4-d]pyrimidin-4-one;
6-{[(3S)-1-(cyclopropylcarbonyl)-3-pyrrolidinyl]methyl}-5-[4-(1H-indol-5-yl)phenyl]-1-methyl-1,5-dihydro-4H-pyrazolo[3,4-d]pyrimidin-4-one;
6-[4-(1-benzofuran-5-yl)-2-fluorophenyl]-5-{[(3R)-1-(cyclopropylcarbonyl)-3-pyrrolidinyl]methyl}-1-methyl-1,5-dihydro-4H-pyrazolo[3,4-d]pyrimidin-4-one;
5-{[(3R)-1-(cyclopropylcarbonyl)-3-pyrrolidinyl]methyl}-6-(3,4′-difluoro-4-biphenylyl)-1-methyl-1,5-dihydro-4H-pyrazolo[3,4-d]pyrimidin-4-one;
5-{[(3R)-1-(cyclopropylcarbonyl)-3-pyrrolidinyl]methyl}-6-[2-fluoro-4-(1H-indol-5-yl)phenyl]-1-methyl-1,5-dihydro-4H-pyrazolo[3,4-d]pyrimidin-4-one;
5-{[(3R)-1-(cyclopropylcarbonyl)-3-pyrrolidinyl]methyl}-6-[2-fluoro-4-(1H-indol-6-yl)phenyl]-1-methyl-1,5-dihydro-4H-pyrazolo[3,4-d]pyrimidin-4-one;
2-[4-(1-benzofuran-5-yl)phenyl]-3-{[(3R)-1-(cyclopropylcarbonyl)-3-pyrrolidinyl]methyl}-4(3H)-quinazolinone;
3-{[(3R)-1-(cyclopropylcarbonyl)-3-pyrrolidinyl]methyl}-2-[4-(1H-indol-6-yl)phenyl]-4(3H)-quinazolinone;
3-{[(3R)-1-(cyclopropylcarbonyl)-3-pyrrolidinyl]methyl}-2-[4-(1H-indol-5-yl)phenyl]-4(3H)-quinazolinone;
3-{[(3R)-1-(cyclopropylcarbonyl)-3-pyrrolidinyl]methyl}-2-[4′-(methyloxy)-4-biphenylyl]-4(3H)-quinazolinone;
2-[2-chloro-4(methoxy)-4-bipheny]-3-{[(3R)-1-(cyclopropylcarbonyl)-3-pyrrolidinyl]methyl}-4(3H)-quinazolinone;
2-[4-(1-benzofuran-5-yl)phenyl]-3-{[(3R)-1-(cyclopropylcarbonyl)-3-pyrrolidinyl]methyl}-6-methyl-4(3H)-pyrimidinone;
3-{[(3R)-1-(cyclopropylcarbonyl)-3-pyrrolidinyl]methyl}-2-[4-(1H-indol-5-yl)phenyl-6-methyl-4(3H)-pyrimidinone;
3-{[(3R)-1-(cyclopropylcarbonyl)-3-pyrrolidinyl]methyl}-2-[4-(1H-indol-6-yl)phenyl-6-methyl-4(3H)-pyrimidinone;
4-({[(3R)-1-(cyclopropylcarbonyl)-3-pyrrolidinyl]methyl}oxy)-6-methyl-2-[4′-(methyloxy)-4-biphenylyl]pyrimidine;
2-[2′-chloro-4′-(methyloxy)-4-biphenylyl]-3-{[(3R)-1-(cyclopropylcarbonyl)-3-pyrrolidinyl]methyl}-6-methyl-4(3H)-pyrimidinone;
N′-[4′-(1-{[(3R)-1-(cyclopropylcarbonyl)-3-pyrrolidinyl]methyl}-4-methyl-6-oxo-1,6-dihydro-2-pyrimidinyl)-3-biphenylyl]-N,N-dimethylsulfamide;
3-{[(3R)-1-(cyclopropylcarbonyl)-3-pyrrolidinyl]methyl}-2-(4′-fluoro-4-biphenylyl)-6-methyl-4(3H)-pyrimidinone;
2-[4-(1-benzofuran-5-yl)-2-fluorophenyl]-3-{[(3R)-1-(cyclopropylcarbonyl)-3-pyrrolidinyl]methyl}-6-methyl-4(3H)-pyrimidinone;
3-{[(3R)-1-(cyclopropylcarbonyl)-3-pyrrolidinyl]methyl}-2-[2-fluoro-4-(1H-indol-5-yl)phenyl]-6-methyl-4(3H)-pyrimidinone;
3-{[(3R)-1-(cyclopropylcarbonyl)-3-pyrrolidinyl]methyl}-2-[2-fluoro-4-(1H-indol-6-yl)phenyl]-6-methyl-4(3H)-pyrimidinone;
3-{[(3R)-1-(cyclopropylcarbonyl)-3-pyrrolidinyl]methyl}-2-(3,4′-difluoro-4-biphenylyl)-6-methyl-4(3H)-pyrimidinone;
3-{[(3R)-1-(cyclopropylcarbonyl)-3-pyrrolidinyl]methyl}-5,6-dimethyl-2-[4′-(methyloxy)-4-biphenylyl]-4(3H)-pyrimidinone;
2-[4-(1-benzofuran-5-yl)phenyl]-3-{[(3R)-1-(cyclopropylcarbonyl)-3-pyrrolidinyl]methyl}-5,6-dimethyl-4(3H)-pyrimidinone;
3-{[(3R)-1-(cyclopropylcarbonyl)-3-pyrrolidinyl]methyl}-2-[4-(1H-indol-6-yl)phenyl]-5,6-dimethyl-4(3H)-pyrimidinone;
3-{[(3R)-1-(cyclopropylcarbonyl)-3-pyrrolidinyl]methyl}-2-(4′-fluoro-4-biphenylyl)-5,6-dimethyl-4(3H)-pyrimidinone;
3-{[(3R)-1-(cyclopropylcarbonyl)-3-pyrrolidinyl]methyl}-2-[2-fluoro-4-(1H-indol-6-yl)phenyl]-5,6-dimethyl-4(3H)-pyrimidinone;
2-[4-(1-benzofuran-5-yl)-2-fluorophenyl]-3-{[(3R)-1-(cyclopropylcarbonyl)-3-pyrrolidinyl]methyl}-5,6-dimethyl-4 (3H)-pyrimidinone;
3-{[(3R)-1-(cyclopropylcarbonyl)-3-pyrrolidinyl]methyl}-2-(3,4′-difluoro-4-biphenylyl)-5,6-dimethyl-4(3H)-pyrimidinone;
3-{[(3R)-1-(cyclopropylcarbonyl)-3-pyrrolidinyl]methyl}-5-ethyl-2-[4-(1H-indol-6-yl)phenyl]-6-methyl-4(3H)-pyrimidinone;
2-[4-(1-benzofuran-5-yl)phenyl]-3-{[(3R)-1-(cyclopropylcarbonyl)-3-pyrrolidinyl]methyl}-5-ethyl-6-methyl-4(3H)-pyrimidinone;
3-{[(3R)-1-(cyclopropylcarbonyl)-3-pyrrolidinyl]methyl}-5-ethyl-2-[4-(1H-indol-5-yl)phenyl]-6-methyl-4(3H)-pyrimidinone;
3-{[(3R)-1-(cyclopropylcarbonyl)-3-pyrrolidinyl]methyl}-5-ethyl-2-(4′-fluoro-4-biphenylyl)-6-methyl-4(3H)-pyrimidinone;
2-{[(3S)-1-(cyclopropylcarbonyl)-3-pyrrolidinyl]methyl}-1-[4-(1H-indol-6-yl)phenyl]-6-methyl-4(3H)-pyrimidinone;
2-{[(3S)-1-(cyclopropylcarbonyl)-3-pyrrolidinyl]methyl}-1-[4-(1H-indol-6-yl)phenyl]-6-methyl-4(1H)-pyrimidinone; and
1-[4-(1-benzofuran-5-yl)phenyl]-2-{[(3S)-1-(cyclopropylcarbonyl)-3-pyrrolidinyl]methyl}-6-methyl-4(1H)-pyrimidinone.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. In general, the salts are formed from pharmaceutically acceptable inorganic and organic acids. More specific examples of suitable acid salts include maleic, hydrochloric, hydrobromic, sulphuric, phosphoric, nitric, perchloric, fumic, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methansulfonic (mesylate), naphthalene-2-sulfonic, benzenesulfonic, hydroxynaphthoic, hydroiodic, malic, teroic, tannic, and the like.

Other representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, oxalate, pamoate (embonate), palmitate, pantothenate, phosphate/diphosphate, polygalacturonate, salicylate, stearate, subacetate, succinate, sulfate, tannate, tartrate, teoclate, tosylate, triethiodide, and valerate salts.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

The compound of Formula (I) or a salt thereof may exist in stereoisomeric forms (e.g., it contains one or more asymmetric carbon atoms). The individual stereoisomers (enantiomers and diastereomers) and mixtures of these are included within the scope of the present invention. The invention also covers the individual isomers of the compound or salt represented by Formula (I) as mixtures with isomers thereof in which one or more chiral centers are inverted. Likewise, it is understood that a compound or salt of Formula (I) may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. Also included within the scope of the invention are individual isomers of the compound represented by Formula (I), as well as any wholly or partially equilibrated mixtures thereof. The present invention also includes the individual isomers of the compound or salt represented by the Formula (I) as well as mixtures with isomers thereof in which one or more chiral centers are inverted. It is to be understood that the present invention includes all combinations and subsets of the particular groups defined hereinabove.

This invention also relates to pharmaceutical compositions comprising a compound of Formula (I), Formula (I)(A), or Formula (I)(B) or pharmaceutically acceptable salt thereof and a pharmaceutically acceptable carrier.

This invention also relates to methods of treating cancer comprising administering to a human in need thereof an effective amount of Formula (I), Formula (I)(A), or Formula (I)(B) or pharmaceutically acceptable salt thereof or a pharmaceutical composition comprising Formula (I), Formula (I)(A), or Formula (I)(B) or pharmaceutically acceptable salt thereof.

This invention also relates to the use of Formula (I), Formula (I)(A), or Formula (I)(B) or pharmaceutically acceptable salt thereof for the treatment of cancer.

In some embodiments the cancer is selected from the group consisting of: brain (gliomas), glioblastomas, leukemias, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, renal, ovarian, pancreatic, prostate, sarcoma, osteosarcoma, giant cell tumor of bone and thyroid.

This invention also relates to methods of treating cancer in a mammal in need thereof, comprising administering to said mammal a therapeutically effective amount of a compound of Formula (I) Formula (I)(A), or Formula (I)(B) or pharmaceutically acceptable salt thereof and at least one anti-neoplastic agent.

### DEFINITIONS

Terms are used within their accepted meanings The following definitions are meant to clarify, but not limit, the terms defined.

As used herein, the term “alkyl” (or “alkylene”) refers to a straight or branched chain alkyl, preferably having from one to twelve carbon atoms, which may be unsubstituted or substituted, saturated or unsaturated with multiple degrees of substitution included within the present invention. Suitable substituents are selected from the group consisting of halogen, amino, cyano, hydroxyl, alkoxy, alkylthio, alkylsulfonyl, amidosulfonyl, oxazole and methylisoxazole. Examples of “alkyl” as used herein include methyl, ethyl, propyl, isopropyl, isobutyl, n-butyl, t-butyl, isopentyl, n-pentyl, and the like, as well as substituted versions thereof.

As used herein, the term “cycloalkyl” refers to an unsubstituted or substituted mono- or polycyclic non-aromatic saturated ring. Exemplary “cycloalkyl” groups include, but are not limited to, cyclopropyl, cyclobutyl, cyclopentyl, cyclohexyl, cycloheptyl, and the like.

As used herein, the term “heterocycloalkyl” refers to a mono- or polycyclic non-aromatic saturated ring containing one or more heteroatoms. Examplary “C₄-C₆heterocycloalkyl” groups include, but are not limited to, azetidinyl, oxetanyl, thietanyl, pyrrolidinyl, tetrahydrofuranyl, tetrahydrothiopenyl, piperidinyl, pyrazolidyinyl, 3-dioxolanyl, morpholinyl, piperazinyl, imidazolidinyl, 1,4-dioxinyl, 1,4-dioxanyl, thiomorpholinyl, thiazolidinyl, isoxazolidinyl, 1,4-oxathianyl, 1,4-dithianyl, trithianyl, 1,3,5-trioxanyl, and 1,3,5-trithianyl and the like.

As used herein, the term “heterocyclyl” refers to a mono- or polycyclic ring system containing one or more heteroatoms. Preferred heteroatoms include nitrogen, oxygen, and sulfur, including N-oxides, sulfur oxides, and dioxides. The term “9- or 10-membered heterocyclyl” represents a fully unsaturated or partially unsaturated, bicyclic group, containing 9 or 10 ring atoms, including 1 to 5 heteroatoms independently selected from nitrogen, oxygen and sulfur. Selected 9- or 10-membered heterocycyl groups contain one nitrogen, oxygen or sulfur ring heteroatom, and optionally contain 1, 2, 3, or 4 additional nitrogen ring atoms and/or 1 additional oxygen or sulfur atom. Examples of 9- or 10-membered heterocyclyl groups include, but are not limited to benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzothiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzothiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

As used herein, the term “aryl,” unless otherwise defined, is meant aromatic, hydrocarbon ring system. The ring system may be monocyclic or fused polycyclic (e.g., bicyclic, tricyclic, etc.). In various embodiments, the monocyclic aryl ring is phenyl. In various embodiments, the polycyclic ring is a bicyclic aryl group. A naphthyl ring, which has 10 carbon atoms, is a suitable polycyclic aryl group.

As used herein, the term “heteroaryl,” unless otherwise defined, is meant an aromatic ring system containing carbon(s) and at least one heteroatom. Heteroaryl may be monocyclic or polycyclic. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junctions, for example, bicyclic heteroaryl is a polycyclic heteroaryl. Bicyclic heteroaryl rings may contain from 9 to 12 member atoms. Monocyclic heteroaryl rings may contain from 5 to 8 member atoms (carbons and heteroatoms).

The 5-membered heteroaryl groups present in the compounds of this invention contain one nitrogen, oxygen, or sulfur ring heteroatom, and optionally contain 1, 2, or 3 additional nitrogen ring atoms. The 6-membered heteroaryl groups present in the compounds of this invention contain 1, 2, 3, or 4 nitrogen ring heteroatoms. Examples of 5- or 6-membered heteroaryl groups include, but are not limited to furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl.

As used herein, the term “cyano” refers to the group —CN.

As used herein, the term “optionally” means that the subsequently described event(s) may or may not occur, and includes both event(s) that occur and event(s) that do not occur.

As used herein, unless otherwise defined, the phrase “optionally substituted” or variations thereof denote an optional substitution, including multiple degrees of substitution, with one or more substituent group.

The invention further provides a pharmaceutical composition (also referred to as pharmaceutical formulation) comprising a compound of Formula (I) or pharmaceutically acceptable salt, thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

In accordance with another aspect of the invention there is provided a process for the preparation of a pharmaceutical composition comprising mixing (or admixing) a compound of Formula (I) or salt thereof with at least one excipient.

### Pharmaceutical Compositions

Pharmaceutical compositions may be in unit dose form containing a predetermined amount of active ingredient per unit dose. Such a unit may contain a therapeutically effective dose of the compound of Formula (I) or salt thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound or salt thereof of the invention or the pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound or salt thereof of the invention in a suitably flavoured aqueous solution, while elixirs are prepared through the use of a non-toxic alcoholic vehicle. Suspensions can be formulated by dispersing the compound or salt of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

As used herein, the term “treatment” includes prophylaxis and refers to alleviating the specified condition, eliminating or reducing one or more symptoms of the condition, slowing or eliminating the progression of the condition, and preventing or delaying the reoccurrence of the condition in a previously afflicted or diagnosed patient or subject. Prophylaxis (or prevention or delay of disease onset) is typically accomplished by administering a drug in the same or similar manner as one would to a patient with the developed disease or condition.

The present invention provides a method of treatment in a mammal, especially a human, suffering from disease conditions targeted by the present compounds. Such treatment comprises the step of administering a therapeutically effective amount of a compound of Formula (I) or salt thereof to said mammal, particularly a human. Treatment can also comprise the step of administering a therapeutically effective amount of a pharmaceutical composition containing a compound of Formula (I) or salt thereof to said mammal, particularly a human.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. Additionally, the active ingredient may be presented as a pharmaceutical composition.

While it is possible that, for use in therapy, a therapeutically effective amount of a compound of Formula (I) or salt thereof may be administered as the raw chemical, it is typically presented as the active ingredient of a pharmaceutical composition or formulation.

The precise therapeutically effective amount of a compound or salt thereof of the invention will depend on a number of factors, including, but not limited to, the age and weight of the subject (patient) being treated, the precise disorder requiring treatment and its severity, the nature of the pharmaceutical formulation/composition, and route of administration, and will ultimately be at the discretion of the attending physician or veterinarian. Typically, a compound of Formula (I) or salt thereof will be given for the treatment in the range of about 0.1 to 100 mg/kg body weight of recipient (patient, mammal) per day and more usually in the range of 0.1 to 10 mg/kg body weight per day. Acceptable daily dosages may be from about 1 to about 1000 mg/day, and preferably from about 1 to about 100 mg/day. This amount may be given in a single dose per day or in a number (such as two, three, four, five, or more) of sub-doses per day such that the total daily dose is the same. An effective amount of a salt thereof may be determined as a proportion of the effective amount of the compound of Formula (I) per se. Similar dosages should be appropriate for treatment (including prophylaxis) of the other conditions referred herein for treatment. In general, determination of appropriate dosing can be readily arrived at by one skilled in medicine or the pharmacy art.

### Combinations

When a compound of Formula (I) is administered for the treatment of cancer, the term “co-administering” and derivatives thereof as used herein is meant either simultaneous administration or any manner of separate sequential administration of a FAS inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present FAS inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. (J. Am. Chem., Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986,” Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine[R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine[1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloro ethyl)amino] tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,34bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S, 10 S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-O—(R)-thenylidene-13-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-Fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G 1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl) methyl]methylamino] benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I—DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1, 2-1)] quino line-3,14-(4H,12H)— dione monohydro chloride, is commercially available as the injectable solution HYCAMTIN®. Topotecan is a derivative of camptothecin which binds to the topoisomerase I—DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

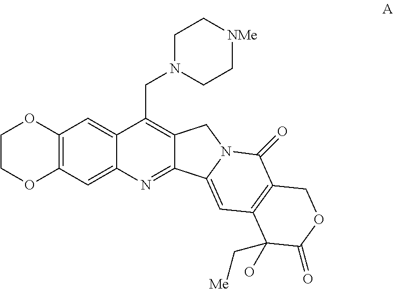 [SMILES: CCC1(O)C(=O)OCc2c1cc1n(c2=O)Cc2c-1nc1cc3c(cc1c2CN1CCN(C)CC1)OCCO3]

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor—I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets,” New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta) IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P.A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R.T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2Activity by a monoclonal Anti-VEGF antibody blocks tumor growth in mice, Cancer Res. (2000) 60, 5117-5124).

Non-receptor kinase angiogenesis inhibitors may also find use in the present invention Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alpha beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the cancer treatment method of the claimed invention includes the co-administration a compound of Formula (I) and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

### EXPERIMENTALS

### Preparation

The derivatives described herein were prepared by the general methods described below:

Schemes:

N-Alkyl pyrazolopyrimidinones can be prepared as outlined in Scheme I. Benzoylation of the 5-amino pyrazole and saponification of the adjacent ester can provide an intermediate acid that can be used for amide bond formation. Deprotection and acylation of the pyrrolidine can provide functionalized precursors that can be cyclized to pyrazolopyimidinone intermediates. Metal mediated cross-coupling reactions can afford the test compounds.

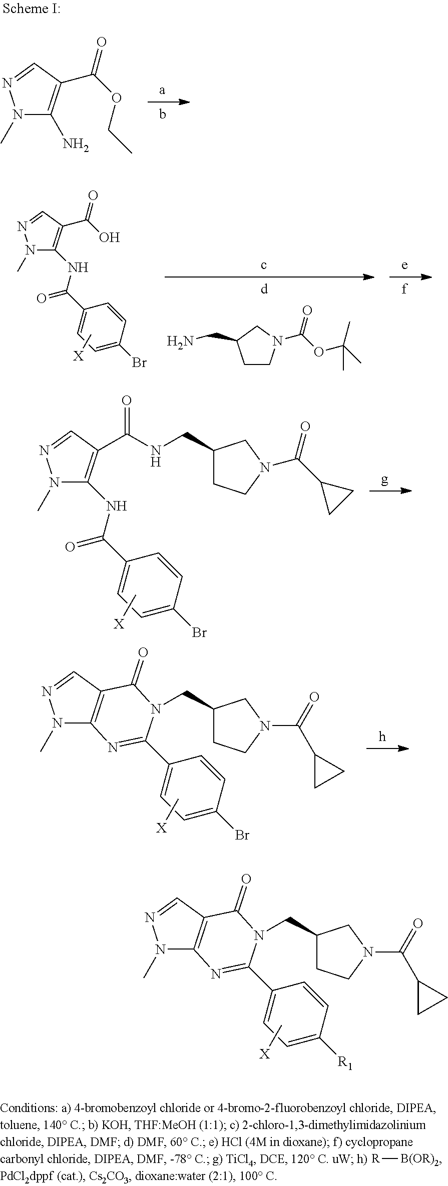

N-Alkyl substituted oxazolopyrimidinones can be prepared as outlined in Scheme II. The same sequence of transformations as shown in Scheme I can be carried out with the appropriate starting materials.

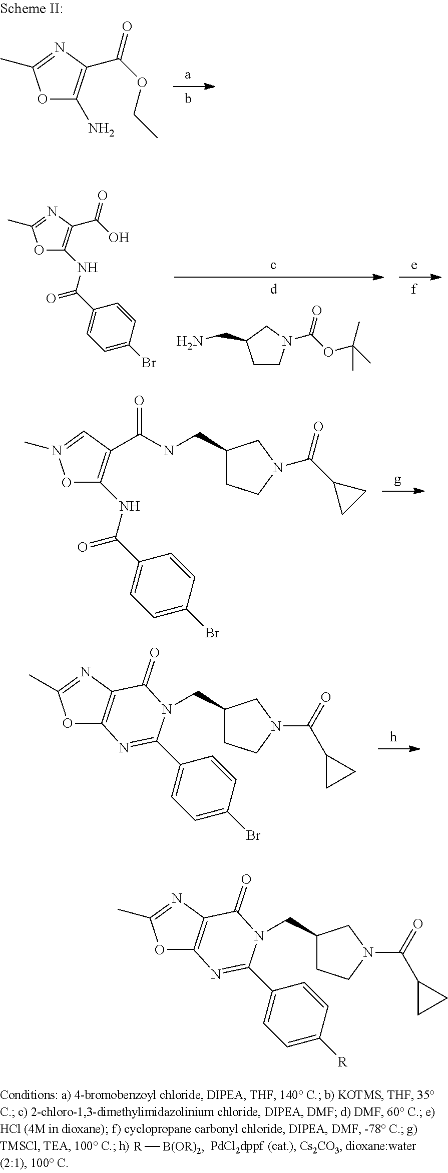

N-Aryl pyrazolopyrimidinones can be prepared as outlined in Scheme III. Deprotection and pyrrolidine functionalization can be carried out after amide bond coupling of the 5-amino pyrazole and chiral carboxylic acid. Saponification and acid activation can provide an intermediate to be used in an amide bond formation reaction with 4-bromoaniline. Ring-closing condensation and metal-mediated coupling can afford the final products.

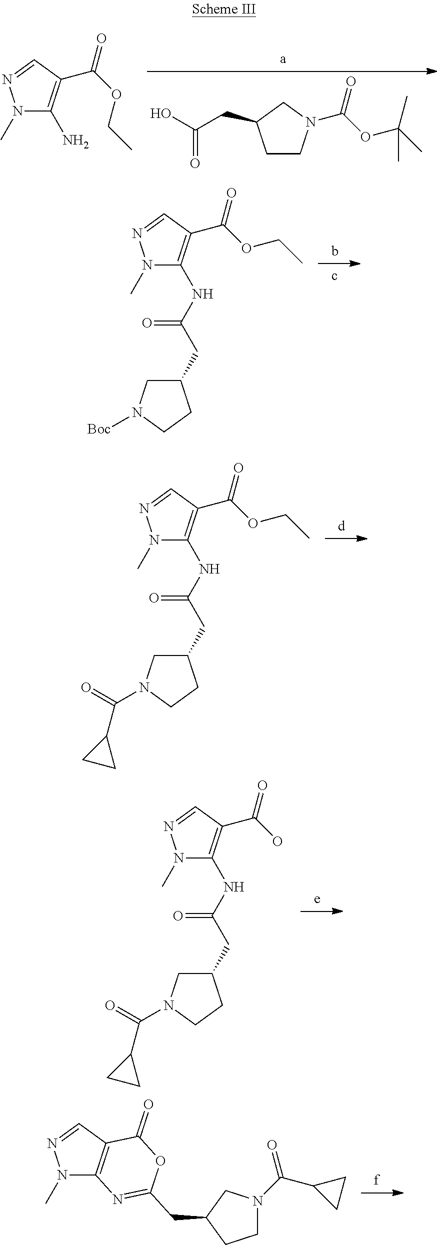

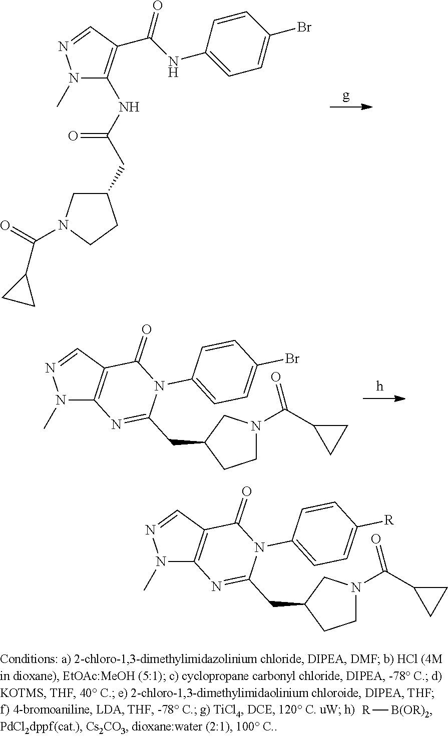

Substituted quinazolinones can be prepared as outlined in Scheme IV. Amide bond formation, ester hydrolysis and Ac₂O mediated cyclization can provide oxazinone intermediates which can be opened with the appropriate amines. Quinazolinone core formation under basic conditions and subsequent metal-mediated coupling can afford the final products.

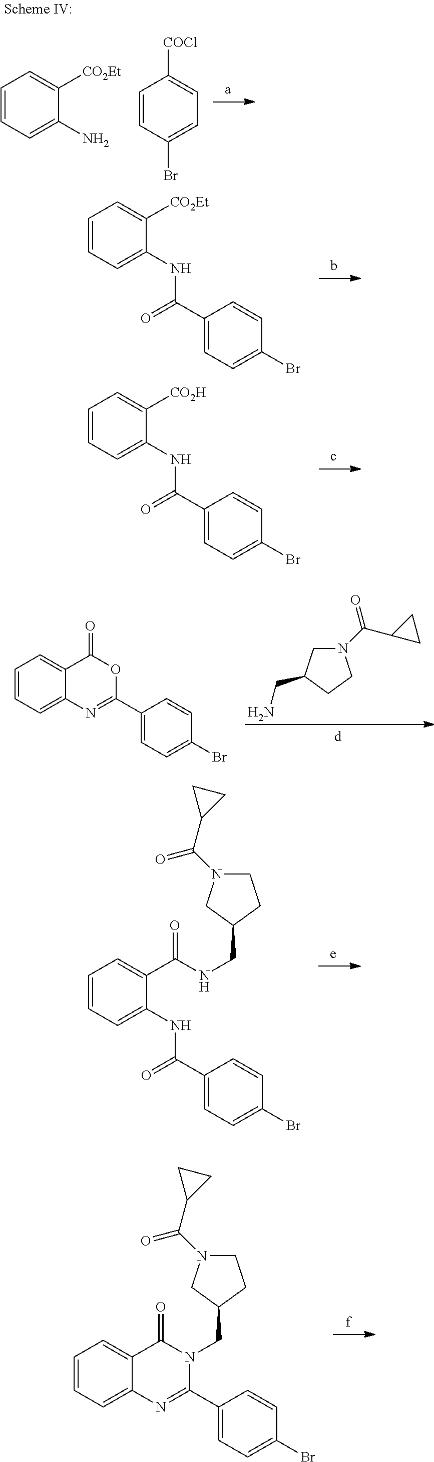

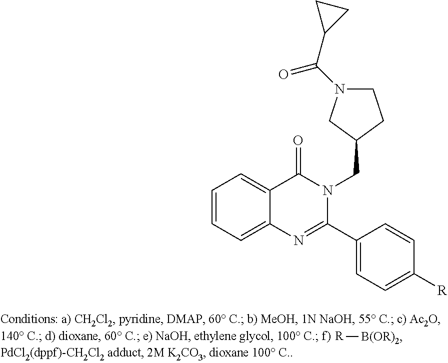

Substituted 6-methylpyrimidinones can be prepared as outlined in Scheme V through titanium (IV) isopropoxide mediate cyclization of benzamide and 3-oxobutan amide intermediates and subsequent metal-mediated couplings.

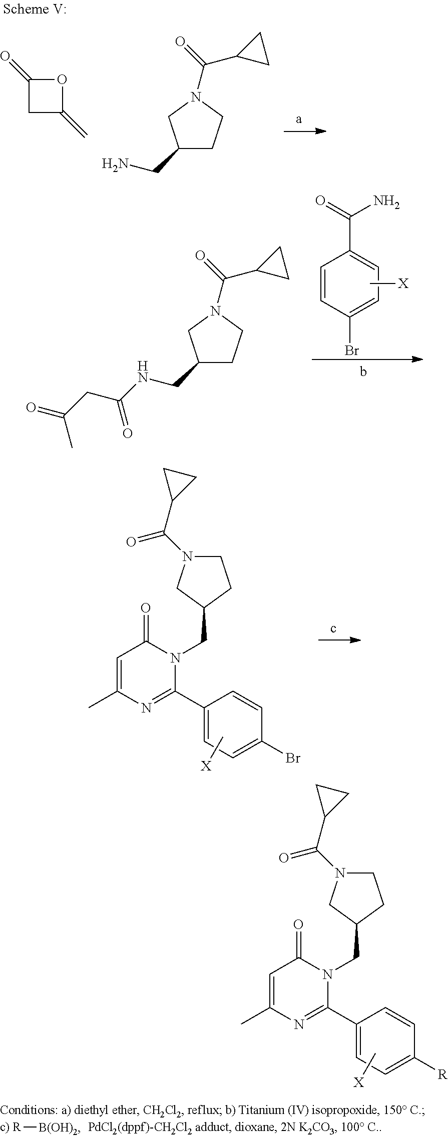

Substituted 5,6-dimethylpyrimidinones and 5-ethyl-6-methylpyrimidinones can be prepared as outlined in Scheme VI through titanium (IV) isopropoxide mediate cyclization of benzamide and 3-oxobutanamide intermediates and subsequent metal-mediated couplings.

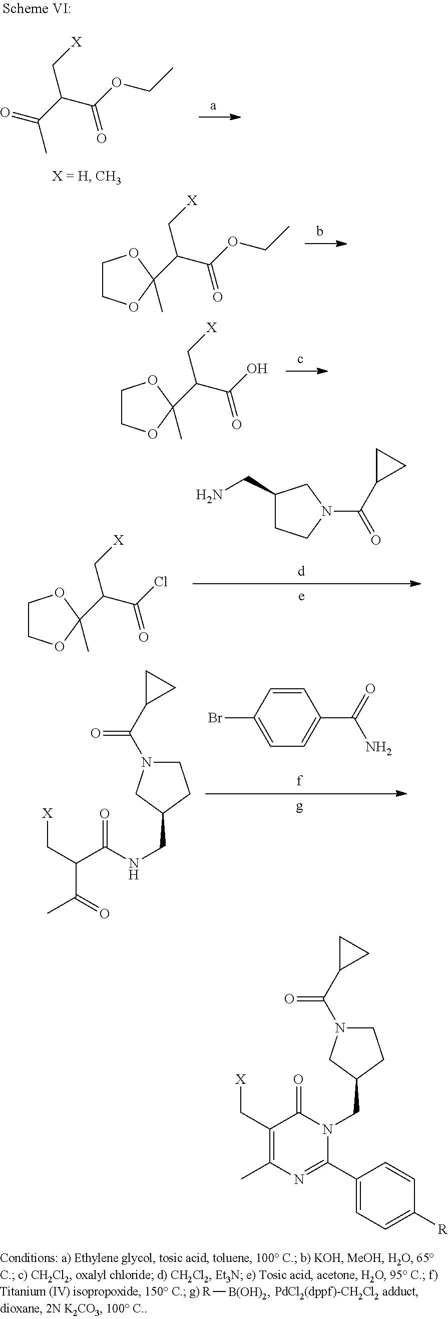

Substituted 4(1H)— and 4(3H)-pyrimidinones can be prepared as outlined in Scheme VII through condensation of (1R)-1-phenylethanamine and itaconic acid followed by conversion of the resulting ester to an amidine. Cyclization of the amidine intermediate can occur with ethyl 2-butynoate under thermal conditions in the presence of N,N-diisopropylethylamine to provide a mixture of the 4(1H)— and 4(3H)-pyrimidinones that can be converted to the test compounds by metal-mediated couplings with aryl boronate esters or acids.

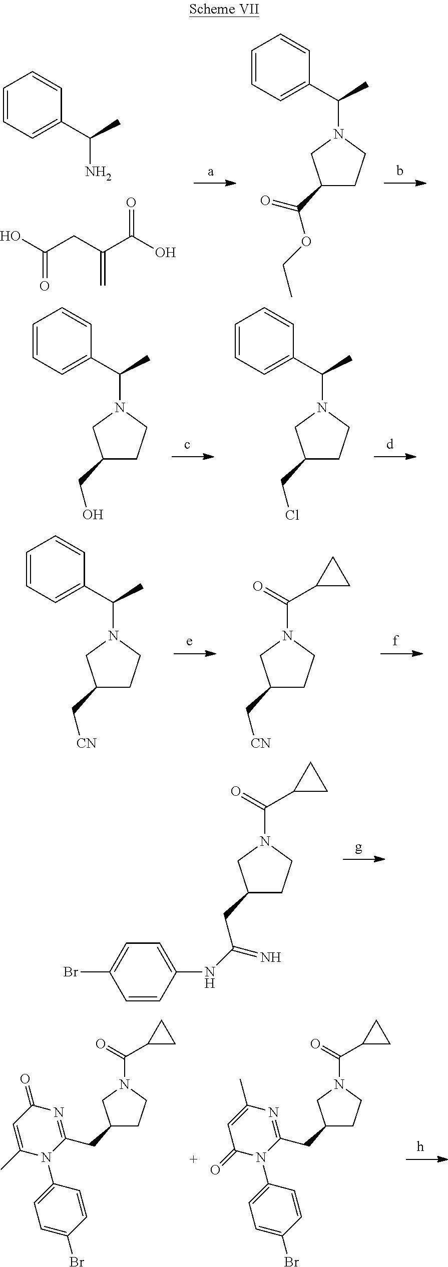

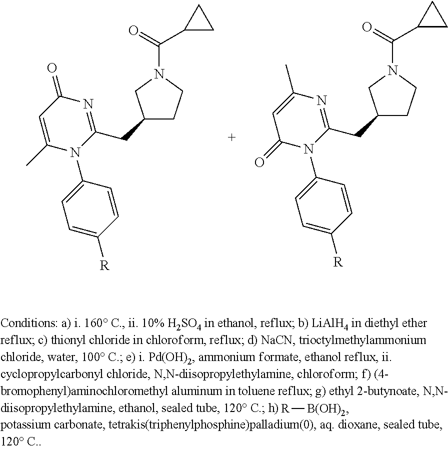

### Experimental Section

The following examples illustrate various non-limiting aspects of this invention.

### Example 1

### 5-{[(3R)-1-(cyclopropylcarbonyl)-3-pyrrolidinyl]methyl}-6-[4-(1H-indol-5-yl)phenyl]-1-methyl-1,5-dihydro-4H-pyrazolo[3,4-d]pyrimidin-4-one

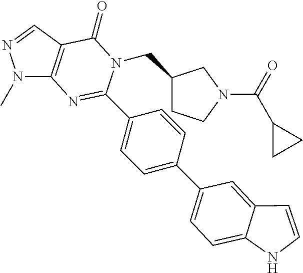 [SMILES: Cn1ncc2c(=O)n(C[C@@H]3CCN(C(=O)C4CC4)C3)c(-c3ccc(-c4ccc5[nH]ccc5c4)cc3)nc21]

### a) Ethyl 5-{[(4-bromophenyl)carbonyl]amino}-1-methyl-1H-pyrazole-4-carboxylate

To a round-bottom flask were added ethyl 5-amino-1-methyl-1H-pyrazole-4-carboxylate (700 mg, 4.14 mmol), 4-bromobenzoyl chloride (1028 mg, 4.68 mmol), toluene (17 mL) and Hunig's base (1.8 ml, 10.31 mmol) under nitrogen. This mixture was refluxed at 140° C. for two hours. LCMS analysis indicated predominantly desired product. The reaction was diluted with EtOAc, poured into a separatory funnel and partitioned with saturated aqueous sodium bicarbonate. The aqueous layer was extracted with EtOAc (3×) and the combined organic layers were dried over Na₂SO₄, filtered, and concentrated under reduced pressure. The residue was purified by silica gel chromatography (40 g, 0-30%, then 30-100% EtOAc/hexane gradient, 50 min). The desired fractions were combined and concentrated to afford ethyl 5-{[(4-bromophenyl)carbonyl]amino}-1-methyl-1H-pyrazole-4-carboxylate as an off-white solid (1050 mg, 72%). LCMS (ES⁺) m/e 352.1, 354.0 [M+H]⁺.

### b) 5-{[(4-Bromophenyl)carbonyl]amino}-1-methyl-1H-pyrazole-4-carboxylic acid

To a round-bottom flask were added ethyl 5-{[(4-bromophenyl)carbonyl]amino}-1-methyl-1H-pyrazole-4-carboxylate (850 mg, 2.414 mmol), KOH (1100 mg, 19.61 mmol), THF (3 mL) and MeOH (3 mL). The mixture was stirred at room temperature for 3 hours. LCMS analysis indicated nearly complete conversion to desired product. 1M HCl was added to the reaction vessel and a precipitate formed. The precipitate was collected via vacuum filtration and dried to afford 5-{[(4-bromophenyl)carbonyl]amino}-1-methyl-1H-pyrazole-4-carboxylic acid as a white solid (700 mg, 90%). LCMS (ES⁺) m/e 324.0, 325.9 [M+H]⁺.

### c) 5-{[(4-Bromophenyl)carbonyl]amino}-N-{[(3S)-1-(cyclopropylcarbonyl)-3-pyrrolidinyl]methyl}-1-methyl-1H-pyrazole-4-carboxamide

To a round-bottom flask were added 5-{[(4-bromophenyl)carbonyl]amino}-1-methyl-1H-pyrazole-4-carboxylic acid (675 mg, 2.082 mmol), 2-chloro-1,3-dimethylimidazolinium chloride (580 mg, 3.41 mmol) and DMF (20 mL) and the mixture was cooled to −78° C. under nitrogen. Hunig's base (0.727 mL, 4.16 mmol) was added to the cooled solution and the mixture was allowed to warm to room temperature. After 30 minutes, 1,1-dimethylethyl (3S)-3-(aminomethyl)-1-pyrrolidinecarboxylate (800 mg, 4.0 mmol) in DMF (2 mL) was added and the mixture was heated at 60° C. overnight. LCMS analysis indicated conversion to the desired intermediate. HCl (10.41 mL, 41.6 mmol) (4M in dioxane) was added and the resulting mixture was heated at 50° C. for 5 hours. LCMS analysis indicated conversion to the desired de-protected intermediate. The mixture was cooled to −78° C. and Hunig's base (11 mL, 63.0 mmol) and cyclopropanecarbonyl chloride (440 mg, 4.21 mmol) in DMF (1 mL) were added. The mixture was allowed to warm to ambient temperature and stirred for 2 hours. LCMS analysis indicated conversion to desired product. The mixture was concentrated, dissolved in EtOAc and poured into a separatory funnel containing water. The aqueous layer was extracted with EtOAc (3×) and the combined organic layers were dried over Na₂SO₄, filtered, and concentrated to an oil. The residue was purified by silica gel chromatography (80 g, 5-40% IPA/EtOAc gradient, 35 min). The desired fractions were combined and concentrated to afford 5-{[(4-bromophenyl)carbonyl]amino}-N-{[(3S)-1-(cyclopropylcarbonyl)-3-pyrrolidinyl]methyl}-1-methyl-1H-pyrazole-4-carboxamide (765 mg, 78%) as an off-white solid. LCMS (ES⁺) m/e 474.0, 475.7 [M+H]⁺.

### d) 6-(4-Bromophenyl)-5-{[(3R)-1-(cyclopropylcarbonyl)-3-pyrrolidinyl]methyl}-1-methyl-1,5-dihydro-4H-pyrazolo[3,4-d]pyrimidin-4-one

To a microwave vial were added 5-{[(4-bromophenyl)carbonyl]amino}-N-{[(3S)-1-(cyclopropylcarbonyl)-3-pyrrolidinyl]methyl}-1-methyl-1H-pyrazole-4-carboxamide (750 mg, 1.581 mmol) and dry 1,2-dichloroethane (DCE) (13.4 mL) under nitrogen. TiCl₄ (6.34 mL, 57.5 mmol) (1M in DCM) was added and the resulting solution was irradiated in a Biotage microwave initiator at 120° C. for 60 minutes. LCMS analysis indicated conversion to desired product. The solution was diluted with DCE and partitioned with saturated NH₄Cl. The aqueous layer was extract with DCE (3×) and the combined organics were dried over Na₂SO₄, filtered and concentrated. The residue was purified by silica gel chromatography (40 g, 5-30% IPA/EtOAc gradient, 35 min). The desired fractions were combined and concentrated to afford 6-(4-bromophenyl)-5-{[(3R)-1-(cyclopropylcarbonyl)-3-pyrrolidinyl]methyl}-1-methyl-1,5-dihydro-4H-pyrazolo[3,4-d]pyrimidin-4-one (502 mg, 70%) as an off-white solid. LCMS (ES⁺) m/e 455.9, 458.0 [M+H]⁺.

### e) 5-{[(3R)-1-(Cyclopropylcarbonyl)-3-pyrrolidinyl]methyl}-6-[4-(1H-indol-5-yl)phenyl]-1-methyl-1,5-dihydro-4H-pyrazolo[3,4-d]pyrimidin-4-one

To a microwave vial were added 6-(4-bromophenyl)-5-{[(3R)-1-(cyclopropylcarbonyl)-3-pyrrolidinyl]methyl}-1-methyl-1,5-dihydro-4H-pyrazolo[3,4-d]pyrimidin-4-one (45 mg, 0.099 mmol), 5-(4,4,5,5-tetramethyl-1,3,2-dioxaborolan-2-yl)-1H-indole (36.0 mg, 0.148 mmol), Cs₂CO₃ (96 mg, 0.296 mmol), and dichloro[1,1′-bis(diphenylphosphino)ferrocene]palladium(II)-dichloromethane adduct (6.0 mg, 0.00735 mmol). 1,4-Dioxane (1 mL) and water (0.5 mL) were added and the mixture was heated overnight at 100° C. LCMS analysis indicated desired product formation and consumption of starting material. The mixture was filtered through a syringe filter and purified by reverse phase HPLC (Gilson, 10-90% MeCN/water+0.1% TFA). The desired fractions were collected and transfered to a reparatory funnel containing EtOAc and saturated aqueous sodium bicarbonate. The aqueous phase was extracted with EtOAc (3×) and the combined organics were washed with brine, dried over Na₂SO₄ and concentrated to afford 5-{[(3R)-1-(cyclopropylcarbonyl)-3-pyrrolidinyl]methyl}-6-[4-(1H-indol-5-yl)phenyl]-1-methyl-1,5-dihydro-4H-pyrazolo[3,4-d]pyrimidin-4-one (39 mg, 80%) as a white solid. LCMS (ES⁺) m/e 493.1 [M+H]⁺.

### Example 2

### N-[4′-(5-{[(3R)-1-(cyclopropylcarbonyl)-3-pyrrolidinyl]methyl}-1-methyl-4-oxo-4,5-dihydro-1H-pyrazolo[3,4-d]pyrimidin-6-yl)-3-biphenylyl]-N,N-dimethylsulfamide

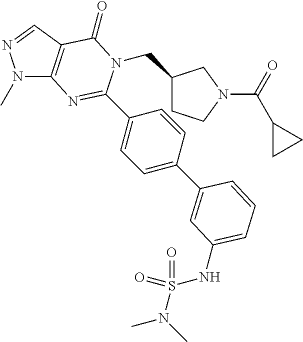 [SMILES: CN(C)S(=O)(=O)Nc1cccc(-c2ccc(-c3nc4c(cnn4C)c(=O)n3C[C@@H]3CCN(C(=O)C4CC4)C3)cc2)c1]

The procedure described for the preparation of example 1 was followed replacing 5-(4,4,5,5-tetramethyl-1,3,2-dioxaborolan-2-yl)-1H-indole with N,N-dimethyl-N-[3-(4,4,5,5-tetramethyl-1,3,2-dioxaborolan-2-yl)phenyl]sulfamide which provided the title compound as a yellow solid (34 mg, 77%). LCMS (ES⁺) m/e 476.6 [M+H]⁺.

### Example 3

### 5-{[(3R)-1-(cyclopropylcarbonyl)-3-pyrrolidinyl]methyl}-6-[4-(1H-indol-6-yl)phenyl]-1-methyl-1,5-dihydro-4H-pyrazolo[3,4-d]pyrimidin-4-one

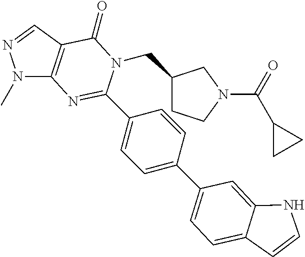 [SMILES: Cn1ncc2c(=O)n(C[C@@H]3CCN(C(=O)C4CC4)C3)c(-c3ccc(-c4ccc5cc[nH]c5c4)cc3)nc21]

The procedure described for the preparation of example 1 was followed replacing 5-(4,4,5,5-tetramethyl-1,3,2-dioxaborolan-2-yl)-1H-indole with 6-(4,4,5,5-tetramethyl-1,3,2-dioxaborolan-2-yl)-1H-indole which provided the title compound as an off-white solid (34 mg, 69%). LCMS (ES⁺) m/e 493.1 [M+H]⁺.

### Example 4

### 6-[4-(1-benzofuran-5-yl)phenyl]-5-{[(3R)-1-(cyclopropylcarbonyl)-3-pyrrolidinyl]methyl}-1-methyl-1,5-dihydro-4H-pyrazolo[3,4-d]pyrimidin-4-one

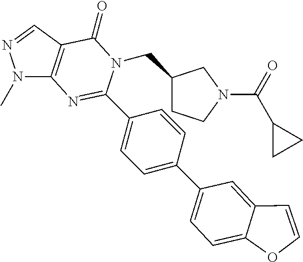 [SMILES: Cn1ncc2c(=O)n(C[C@@H]3CCN(C(=O)C4CC4)C3)c(-c3ccc(-c4ccc5occc5c4)cc3)nc21]

The procedure described for the preparation of example 1 was followed replacing 5-(4,4,5,5-tetramethyl-1,3,2-dioxaborolan-2-yl)-1H-indole with 5-(4,4,5,5-tetramethyl-1,3,2-dioxaborolan-2-yl)-1-benzofuran which provided the title compound as an off-white solid (29 mg, 59%). LCMS (ES⁺) m/e 494.1 [M+H]⁺.

### Example 5

### 5-{[(3R)-1-(cyclopropylcarbonyl)-3-pyrrolidinyl]methyl}-6-[4-(1H-indazol-5-yl)phenyl]-1-methyl-1,5-dihydro-4H-pyrazolo[3,4-d]pyrimidin-4-one

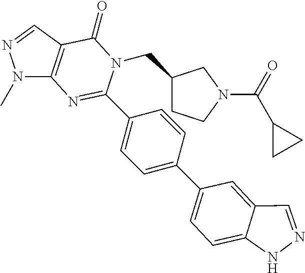 [SMILES: Cn1ncc2c(=O)n(C[C@@H]3CCN(C(=O)C4CC4)C3)c(-c3ccc(-c4ccc5[nH]ncc5c4)cc3)nc21]

The procedure described for the preparation of example 1 was followed replacing 5-(4,4,5,5-tetramethyl-1,3,2-dioxaborolan-2-yl)-1H-indole with 5-(4,4,5,5-tetramethyl-1,3,2-dioxaborolan-2-yl)-1H-indazole which provided the title compound as an off-white solid (17 mg, 35%). LCMS (ES⁺) 494.1 [M+H]⁺.

### Example 6

### 5-{[(3R)-1-(cyclopropylcarbonyl)-3-pyrrolidinyl]methyl}-1-methyl-6-[4-(6-quinolinyl)phenyl]-1,5-dihydro-4H-pyrazolo[3,4-d]pyrimidin-4-one

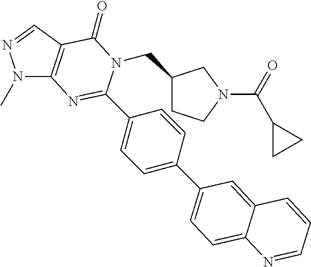 [SMILES: Cn1ncc2c(=O)n(C[C@@H]3CCN(C(=O)C4CC4)C3)c(-c3ccc(-c4ccc5ncccc5c4)cc3)nc21]

The procedure described for the preparation of example 1 was followed replacing 5-(4,4,5,5-tetramethyl-1,3,2-dioxaborolan-2-yl)-1H-indole with 6-(4,4,5,5-tetramethyl-1,3,2-dioxaborolan-2-yl)quinoline which provided the title compound as an off-white solid (43 mg, 97%). LCMS (ES⁺) m/e 505.1 [M+H]⁺.

### Example 7

### 5-{[(3R)-1-(cyclopropylcarbonyl)-3-pyrrolidinyl]methyl}-1-methyl-6-[4-(7-quinolinyl)phenyl]-1,5-dihydro-4H-pyrazolo[3,4-d]pyrimidin-4-one

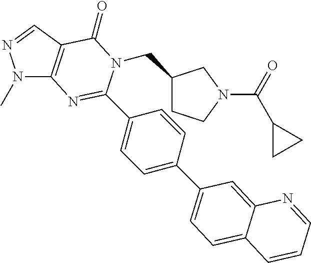 [SMILES: Cn1ncc2c(=O)n(C[C@@H]3CCN(C(=O)C4CC4)C3)c(-c3ccc(-c4ccc5cccnc5c4)cc3)nc21]

The procedure described for the preparation of example 1 was followed replacing 5-(4,4,5,5-tetramethyl-1,3,2-dioxaborolan-2-yl)-1H-indo le with 7-(4,4,5,5-tetramethyl-1,3,2-dioxaborolan-2-yl)quino line which provided the title compound as an off-white solid (7 mg, 16%). LCMS (ES⁺) m/e 505.1 [M+H]⁺.

### Example 8

### 6-[4-(1,3-benzothiazol-5-yl)phenyl]-5-{[(3R)-1-(cyclopropylcarbonyl)-3-pyrrolidinyl]methyl}-1-methyl-1,5-dihydro-4H-pyrazolo[3,4-d]pyrimidin-4-one

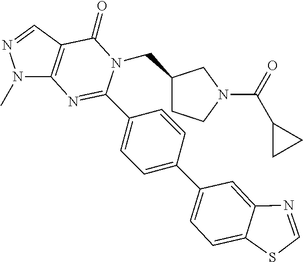 [SMILES: Cn1ncc2c(=O)n(C[C@@H]3CCN(C(=O)C4CC4)C3)c(-c3ccc(-c4ccc5scnc5c4)cc3)nc21]

### a) 5-(4,4,5,5-tetramethyl-1,3,2-dioxaborolan-2-yl)-1,3-benzothiazole

To a microwave vial were added 5-bromo-1,3-benzothiazole (250 mg, 1.168 mmol), bis(pinacolato)diboron (326 mg, 1.285 mmol), potassium acetate (401 mg, 4.09 mmol) and 1,1′-bis(diphenylphosphino)ferrocene-palladium(II)dichloride dichloromethane complex (95 mg, 0.117 mmol) under nitrogen. 1,4-Dioxane (11.7 mL) was added and the mixture was heated overnight at 100° C. LCMS analysis indicated desired product formation. The palladium residue was filtered and washed with EtOAc. The filtrate was partitioned with saturated sodium bicarbonate in a separatory funnel, and the aqueous phase was extracted with EtOAc (3×). The combined organic layers were washed with brine, dried over Na₂SO₄, and concentrated. The residue was purified by silica gel chromatography (40 g, 5-70% EtOAc/Hexane gradient, 30 min). The desired fractions were combined and concentrated to afford the desired product as a black crystalline solid, 5-(4,4,5,5-tetramethyl-1,3,2-dioxaborolan-2-yl)-1,3-benzothiazole (260 mg, 85%). LCMS (ES⁺) m/e 261.9 [M+H]⁺.

### b) 6-[4-(1,3-benzothiazol-5-yl)phenyl]-5-{[(3R)-1-(cyclopropylcarbonyl)-3-pyrrolidinyl]methyl}-1-methyl-1,5-dihydro-4H-pyrazolo[3,4-d]pyrimidin-4-one

The procedure described for the preparation of example 1 was followed replacing 5-(4,4,5,5-tetramethyl-1,3,2-dioxaborolan-2-yl)-1H-indole with 5-(4,4,5,5-tetramethyl-1,3,2-dioxaborolan-2-yl)-1,3-benzothiazole which provided the title compound as an off-white solid (39 mg, 87%). LCMS (ES⁺) m/e 505.1 [M+H]⁺.

### Example 9

### 5-[4-(1-benzofuran-5-yl)phenyl]-6-{[(3R)-1-(cyclopropylcarbonyl)-3-pyrrolidinyl]methyl}-2-methyl[1,3]oxazolo[5,4-d]pyrimidin-7(6H)-one

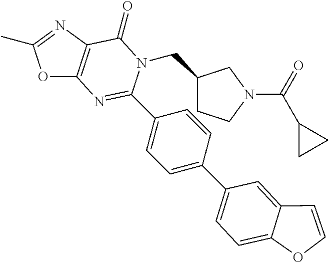 [SMILES: Cc1nc2c(=O)n(C[C@@H]3CCN(C(=O)C4CC4)C3)c(-c3ccc(-c4ccc5occc5c4)cc3)nc2o1]

### a) Ethyl 5-{[(4-bromophenyl)carbonyl]amino}-2-methyl-1,3-oxazole-4-carboxylate

The procedure described for the preparation of example 1a was followed replacing ethyl 5-amino-1-methyl-1H-pyrazole-4-carboxylate with ethyl 5-amino-2-methyl-1,3-oxazole-4-carboxylate which provided the title compound as an off-white solid (650 mg, 15%). LCMS (ES⁺) m/e 353.1, 354.7 [M+H]⁺.

### b) 5-{[(4-Bromophenyl)carbonyl]amino}-2-methyl-1,3-oxazole-4-carboxylic acid

To a round-bottom flask, ethyl 5-{[(4-bromophenyl)carbonyl]amino}-2-methyl-1,3-oxazole-4-carboxylate (600 mg, 1.70 mmol) was dissolved in THF (25 mL) and warmed to 35° C. in an oil bath. KOTMS (1308 mg, 10.19 mmol) was added in 6 portions to the stirred solution over a 20 minute period. LCMS analysis indicated conversion to desired product. The mixture was quenched with saturated NH₄Cl and poured into a separatory funnel containing EtOAc. The aqueous layer was extracted with EtOAc (4×) and the combined organics were washed with brine, dried over Na₂SO₄, filtered, and concentrated to afford 5-{[(4-bromophenyl)carbonyl]amino}-2-methyl-1,3-oxazole-4-carboxylic acid as a tan solid (504 mg, 91%). LCMS (ES⁺) m/e 325.0, 326.9 M+H]⁺.

### c) 5-{[(4-Bromophenyl)carbonyl]amino}-N-{[(3S)-1-(cyclopropylcarbonyl)-3-pyrrolidinyl]methyl}-2-methyl-1,3-oxazole-4-carboxamide

The procedure described for the preparation of example 1c was followed replacing 5-{[(4-bromophenyl)carbonyl]amino}-1-methyl-1H-pyrazole-4-carboxylic acid with 5-{[(4-bromophenyl)carbonyl]amino}-2-methyl-1,3-oxazole-4-carboxylic acid which provided the title compound as a white solid (497 mg, 70%). LCMS (ES⁺) m/e 475.0, 476.9 [M+H]⁺.

### d) 5-(4-Bromophenyl)-6-{[(3R)-1-(cyclopropylcarbonyl)-3-pyrrolidinyl]methyl}-2-methyl[1,3]oxazolo[5,4-d]pyrimidin-7(6H)-one

To a microwave vial were added 5-{[(4-bromophenyl)carbonyl]amino}-N-{[(3S)-1-(cyclopropylcarbonyl)-3-pyrrolidinyl]methyl}-2-methyl-1,3-oxazole-4-carboxamide (425 mg, 0.894 mmol), TMSC1 (2286 μA, 17.88 mmol) and triethylamine (4985 μA, 35.8 mmol) and the resulting solution was heated at 100° C. for 24 hours. LCMS analysis indicated complete conversion to desired product. The solution was diluted with DCM and quenched with 1N HCl (3 mL). This mixture was added to a reparatory funnel containing DCM and water. The aqueous layer was extracted with DCM (3×) and the combined organics were washed with brine, dried over Na₂SO₄, filtered and concentrated. This residue was purified by silica gel chromatography (24 g, 0-15% IPA/EtOAc gradient, 35 min). The desired fractions were combined and concentrated to afford 5-(4-bromophenyl)-6-{[(3R)-1-(cyclopropylcarbonyl)-3-pyrrolidinyl]methyl}-2-methyl[1,3]oxazolo[5,4-d]pyrimidin-7(6H)-one (290 mg, 71%) as a white solid. LCMS (ES⁺) m/e 457.1, 459.2 [M+H]⁺.

### e) 5-[4-(1-Benzofuran-5-yl)phenyl]-6-{[(3R)-1-(cyclopropylcarbonyl)-3-pyrrolidinyl]methyl}-2-methyl[1,3]oxazolo[5,4-d]pyrimidin-7(6H)-one

To a microwave vial were added 5-(4-bromophenyl)-6-{[(3R)-1-(cyclopropylcarbonyl)-3-pyrrolidinyl]methyl}-2-methyl [1,3]oxazolo[5,4-d]pyrimidin-7(6H)-one (40 mg, 0.087 mmol), 5-(4,4,5,5-tetramethyl-1,3,2-dioxaborolan-2-yl)-1-benzofuran (21.35 mg, 0.087 mmol), Cs₂CO₃ (100 mg, 0.306 mmol), dichloro[1,1′-bis(diphenylphosphino)ferrocene]palladium(II)-dichloromethane adduct (6.0 mg, 0.00735 mmol), 1,4-dioxane (1 ml) and water (0.5 ml). The mixture was purged with nitrogen and heated overnight on a hot plate at 100° C. LCMS analysis indicated desired product formation and consumption of starting material. The mixture was filtered through a syringe filter and purified by reverse phase HPLC (Gilson, 10-90% MeCN/water+0.1% TFA). The desired fractions were collected and added to a separatory funnel containing EtOAc and saturated sodium bicarbonate. The aqueous phase was extracted with EtOAc (3×) and the combined organics were washed with brine, dried over Na₂SO₄, and concentrated to afford 5-[4-(1-benzofuran-5-yl)phenyl]-6-{[(3R)-1-(cyclopropylcarbonyl)-3-pyrrolidinyl]methyl}-2-methyl[1,3]oxazolo[5,4-d]pyrimidin-7(6H)-one (12 mg, 27%) as a white solid. LCMS (ES⁺) m/e 495.3 [M+H]⁺.

### Example 10

### 4′-(6-{[(3R)-1-(cyclopropylcarbonyl)-3-pyrrolidinyl]methyl}-2-methyl-7-oxo-6,7-dihydro[1,3]oxazolo[5,4-d]pyrimidin-5-yl)-4-biphenylcarbonitrile

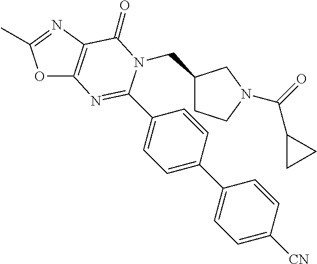 [SMILES: Cc1nc2c(=O)n(C[C@@H]3CCN(C(=O)C4CC4)C3)c(-c3ccc(-c4ccc(C#N)cc4)cc3)nc2o1]

The procedure described for the preparation of example 9 (step e) was followed replacing 5-(4,4,5,5-tetramethyl-1,3,2-dioxaborolan-2-yl)-1-benzofuran with 4-(4,4,5,5-tetramethyl-1,3,2-dioxaborolan-2-yl)benzonitrile which provided the title compound as a white solid (18 mg, 43%). LCMS (ES⁺) 480.1 [M+H]⁺.

### Example 11

### 6-{[(3R)-1-(cyclopropylcarbonyl)-3-pyrrolidinyl]methyl}-5-[4-(1H-indazol-5-yl)phenyl]-2-methyl[1,3]oxazolo[5,4-d]pyrimidin-7(6H)-one

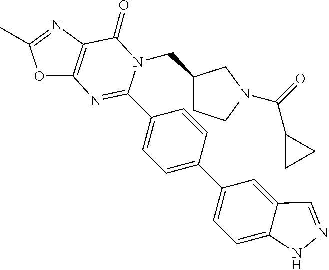 [SMILES: Cc1nc2c(=O)n(C[C@@H]3CCN(C(=O)C4CC4)C3)c(-c3ccc(-c4ccc5[nH]ncc5c4)cc3)nc2o1]

The procedure described for the preparation of example 9 (step e) was followed replacing 5-(4,4,5,5-tetramethyl-1,3,2-dioxaborolan-2-yl)-1-benzofuran with 5-(4,4,5,5-tetramethyl-1,3,2-dioxaborolan-2-yl)-1H-indazole which provided the title compound as a white solid (8 mg, 19%). LCMS (ES⁺) m/e 495.2 [M+H]⁺.

### Example 12

### 5-[4-(1,3-benzothiazol-5-yl)phenyl]-6-{[(3R)-1-(cyclopropylcarbonyl)-3-pyrrolidinyl]methyl}-2-methyl[1,3]oxazolo[5,4-d]pyrimidin-7(6H)-one

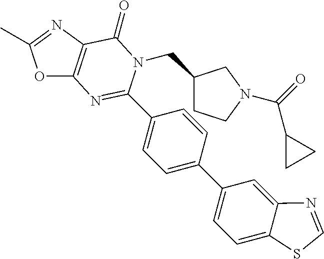 [SMILES: Cc1nc2c(=O)n(C[C@@H]3CCN(C(=O)C4CC4)C3)c(-c3ccc(-c4ccc5scnc5c4)cc3)nc2o1]

The procedure described for the preparation of example 9 (step e) was followed replacing 5-(4,4,5,5-tetramethyl-1,3,2-dioxaborolan-2-yl)-1-benzofuran with 5-(4,4,5,5-tetramethyl-1,3,2-dioxaborolan-2-yl)-1,3-benzothiazole, providing the title compound as a white solid (10 mg, 23%). LCMS (ES⁺) m/e 512.2 [M+H]⁺.

### Example 13

### 6-{[(3R)-1-(cyclopropylcarbonyl)-3-pyrrolidinyl]methyl}-5-[4-(1H-indol-5-yl)phenyl]-2-methyl[1,3]oxazolo[5,4-d]pyrimidin-7(6H)-one

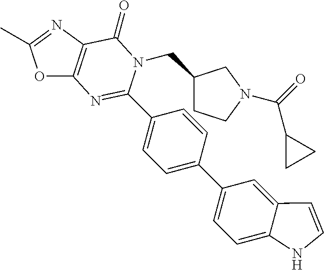 [SMILES: Cc1nc2c(=O)n(C[C@@H]3CCN(C(=O)C4CC4)C3)c(-c3ccc(-c4ccc5[nH]ccc5c4)cc3)nc2o1]

The procedure described for the preparation of example 9 (step e) was followed replacing 5-(4,4,5,5-tetramethyl-1,3,2-dioxaborolan-2-yl)-1-benzofuran with 5-(4,4,5,5-tetramethyl-1,3,2-dioxaborolan-2-yl)-1H-indole, providing the title compound as a white solid (25 mg, 58%). LCMS (ES⁺) m/e 494.2 [M+H]⁺.

### Example 14

### 6-{[(3S)-1-(cyclopropylcarbonyl)-3-pyrrolidinyl]methyl}-5-[4-(1H-indol-6-yl)phenyl]-1-methyl-1,5-dihydro-4H-pyrazolo[3,4-d]pyrimidin-4-one

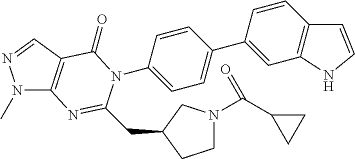 [SMILES: Cn1ncc2c(=O)n(-c3ccc(-c4ccc5cc[nH]c5c4)cc3)c(C[C@@H]3CCN(C(=O)C4CC4)C3)nc21]

### a) Ethyl 5-{[((3R)-1-{[(1,1-dimethylethyl)oxy]carbonyl}-3-pyrrolidinyl)acetyl]amino}-1-methyl-1H-pyrazole-4-carboxylate

To a round bottom flask were added ((3S)-1-{[(1,1-dimethylethyl)oxy]carbonyl}-3-pyrrolidinyl)acetic acid (8.04 g, 35.1 mmol) and 2-chloro-1,3-dimethylimidazolinium chloride (5.95 g, 35.1 mmol) and the flask was purged with nitrogen. DMF (80 mL) and Hunig's base (15.5 mL, 89 mmol) were added and the mixture was stirred at room temperature for 45 minutes. LCMS analysis indicated desired intermediate formation. A solution of ethyl 5-amino-1-methyl-1H-pyrazole-4-carboxylate (5 g, 29.6 mmol) in DMF (20 mL) was slowly added to the solution. After 2 h, the reaction was diluted with EtOAc and partitioned with brine. The aqueous layer was extracted with EtOAc (2×) and the combined organic layers were washed with water, brine (2×), dried over Na₂SO₄ and concentrated to an oil. The residue was purified by reverse phase HPLC (Gilson, 10-90% MeCN/water+0.1% TFA). The desired fractions were collected and concentrated to afford the desired product as a yellow oil, ethyl 5-{[((3R)-1-{[(1,1-dimethylethyl)oxy]carbonyl}-3-pyrrolidinyl)acetyl]amino}-1-methyl-1H-pyrazole-4-carboxylate (2.8 g, 24%). LCMS (ES⁺) m/e 381.2 [M+H]⁺.

### b) Ethyl 5-({[(3S)-1-(cyclopropylcarbonyl)-3-pyrrolidinyl]acetyl} amino)-1-methyl-1H-pyrazole-4-carboxylate

To a round bottom flask were added ethyl 5-{[((3S)-1-{[(1,1-dimethylethyl)oxy]carbonyl}-3-pyrrolidinyl)acetyl]amino}-1-methyl-1H-pyrazole-4-carboxylate (2.8 g, 7.36 mmol), EtOAc (20 mL) and methanol (4 mL). HCl (5.5 mL, 22.08 mmol, 4M in dioxane) was added and the solution was allowed to stir at room temperature. After 4 hours, LCMS analysis indicated consumption of starting material. The solution was concentrated and re-dissolved in DCM (40 mL). This mixture was cooled to −78° C. and Hunig's base (5.14 mL, 29.4 mmol) and cyclopropanecarbonyl chloride (0.74 mL, 8.10 mmol) were added. The mixture was allowed to warm to ambient temperature and stirred for 2 hours. LCMS analysis indicated conversion to desired product. The mixture was concentrated, re-dissolved in EtOAc, and poured into a separatory funnel containing saturated sodium bicarbonate. The aqueous layer was extracted with EtOAc (3×) and the combined organic layers were dried over Na₂SO₄, filtered and concentrated to an oil. The residue was purified by reverse phase HPLC (Gilson, 10-90% MeCN/water+0.1% TFA). The desired fractions were collected and partitioned in a separatory funnel containing EtOAc and saturated sodium bicarbonate. The aqueous layer was extracted with EtOAc (3×) and the combined organic layers were dried over Na₂SO₄, filtered and concentrated to afford ethyl 5-({[(3S)-1-(cyclopropylcarbonyl)-3-pyrrolidinyl]acetyl} amino)-1-methyl-1H-pyrazole-4-carboxylate (1.25 g, 49%) as an off-white solid. LCMS (ES⁺) m/e 349.1 [M+H]⁺.

### c) 5-({[3S)-1-(Cyclopropylcarbonyl)-3-pyrrolidinyl]acetyl} amino)-1-methyl-1H-pyrazole-4-carboxylic acid

To a round bottom flask were added ethyl 5-({[(3S)-1-(cyclopropylcarbonyl)-3-pyrrolidinyl]acetyl} amino)-1-methyl-1H-pyrazole-4-carboxylate (1.18 g, 3.39 mmol) and THF (28.2 mL). KOTMS (4.34 g, 33.9 mmol) was added portionwise to the reaction mixture over 20 minutes. This mixture was heated at 40° C. for 2 hours. LCMS analysis indicated desired product formation. The solution was concentrated and the residue was dissolved in DMSO and purified by reverse phase HPLC (Gilson, 10-90% MeCN/water+0.1% TFA). The desired fractions were collected and concentrated to afford 5-({[(3S)-1-(cyclopropylcarbonyl)-3-pyrrolidinyl]acetyl} amino)-1-methyl-1H-pyrazole-4-carboxylic acid (1.05 g, 97%) as an off-white solid. LCMS (ES⁺) m/e 494.6 [M+H]⁺.

### d) 6-{[(3S)-1-(Cyclopropylcarbonyl)-3-pyrrolidinyl]methyl}-1-methyl-3a,7a-dihydropyrazolo[3,4-d][1,3]oxazin-4(1H)-one

To a round bottom flask were added 5-({[(3S)-1-(cyclopropylcarbonyl)-3-pyrrolidinyl]acetyl} amino)-1-methyl-1H-pyrazole-4-carboxylic acid (1025 mg, 3.20 mmol), 2-chloro-1,3-dimethylimidazolinium chloride (599 mg, 3.52 mmol) and THF (5 mL). Hunig's base (0.68 mL, 3.84 mmol) was added and the solution was allowed to stir at room temperature for 1 hour. LCMS indicated desired product formation. The solution was diluted with EtOAc and poured into a separatory funnel containing saturated sodium bicarbonate. The aqueous layer was extracted with EtOAc (3×) and the combined organic layers were dried over Na₂SO₄, filtered and concentrated to an oil. This residue was purified by silica gel chromatography (12 g, 20-100% EtOAc/Hexane gradient, 30 min). The desired fractions were combined and concentrated to afford 6-{[(3S)-1-(cyclopropylcarbonyl)-3-pyrrolidinyl]methyl}-1-methyl-3a,7a-dihydropyrazolo[3,4-d][1,3]oxazin-4(1H)-one (260 mg, 27%) as a white solid. LCMS (ES⁺) m/e 303.2 [M+H]⁺.

### e) N-(4-Bromophenyl)-5-({[(3S)-1-(cyclopropylcarbonyl)-3-pyrrolidinyl]acetyl} amino)-1-methyl-1H-pyrazole-4-carboxamide

To a round bottom flask were added 6-{[(3S)-1-(cyclopropylcarbonyl)-3-pyrrolidinyl]methyl}-1-methyl-3a,7a-dihydropyrazolo[3,4-d][1,3]oxazin-4(1H)-one (250 mg, 0.821 mmol) and THF (2 mL) under nitrogen. The mixture was cooled to −78° C. In a separate flask under nitrogen, 4-bromo aniline (268 mg, 1.561 mmol) and dry THF (2 mL) were added. In a third flask, LDA was prepared by dissolving diisopropylamine (0.246 mL, 1.725 mmol) in dry THF (1 mL), cooling to −78° C. and adding n-BuLi (1.027 mL, 1.643 mmol). After 1 h, the prepared LDA was added to the aniline solution at 0° C. and the mixture was allowed to stir for 1 minute. This solution was transferred to the first flask and immediately allowed to warm to room temperature. LCMS analysis indicated conversion to desired product. The mixture was quenched by the addition of saturated sodium bicarbonate and diluted with EtOAc. The aqueous layer was extracted with EtOAc (3×) and the combined organic layers were dried over Na₂SO₄, filtered and concentrated to an oil. The residue was purified by reverse phase HPLC (Gilson, 10-90% MeCN/water+0.1% TFA). The desired fractions were collected and concentrated to afford N-(4-bromophenyl)-5-({[(3S)-1-(cyclopropylcarbonyl)-3-pyrrolidinyl]acetyl} amino)-1-methyl-1H-pyrazole-4-carboxamide (70 mg, 18%) as an off-white solid. LCMS (ES⁺) m/e 474.0, 476.0 [M+H]⁺.

### f) 5-(4-Bromophenyl)-6-{[(3S)-1-(cyclopropylcarbonyl)-3-pyrrolidinyl]methyl}-1-methyl-1,5-dihydro-4H-pyrazolo[3,4-d]pyrimidin-4-one

To a microwave vial were added N-(4-bromophenyl)-5-({[(3S)-1-(cyclopropylcarbonyl)-3-pyrrolidinyl]acetyl} amino)-1-methyl-1H-pyrazole-4-carboxamide (80 mg, 0.169 mmol) and 1,2-dichloroethane (DCE) (1800 μA) under nitrogen. TiCl₄ (675 μA, 0.675 mmol, 1M in DCM) was added and the solution was irradiated in a Biotage microwave initiator at 120° C. for 80 minutes. LCMS analysis indicated conversion to desired product. The solution was diluted with DCE and partitioned with saturated aqueous NH₄Cl. The aqueous layer was extracted with DCE (3×) and the combined organic layers were dried over Na₂SO₄, filtered and concentrated to afford an off-white solid. This residue was purified by silica gel chromatography (24 g, 5-30% IPA/EtOAc gradient, 30 min). The desired fractions were combined and concentrated to afford 5-(4-bromophenyl)-6-{[(3S)-1-(cyclopropylcarbonyl)-3-pyrrolidinyl]methyl}-1-methyl-1,5-dihydro-4H-pyrazolo[3,4-d]pyrimidin-4-one (57 mg, 74%) as a white solid. LCMS (ES⁺) 456.0, 458.2 [M+H]⁺.

### g) 6-{[(3S)-1-(Cyclopropylcarbonyl)-3-pyrrolidinyl]methyl}-5-[4-(1H-indol-6-yl)phenyl]-1-methyl-1,5-dihydro-4H-pyrazolo[3,4-d]pyrimidin-4-one

To a microwave vial were added 5-(4-bromophenyl)-6-{[(3S)-1-(cyclopropylcarbonyl)-3-pyrrolidinyl]methyl}-1-methyl-1,5-dihydro-4H-pyrazolo[3,4-d]pyrimidin-4-one (35 mg, 0.077 mmol), 6-(4,4,5,5-tetramethyl-1,3,2-dioxaborolan-2-yl)-1H-indole (28.0 mg, 0.115 mmol), Cs₂CO₃ (87 mg, 0.268 mmol) and dichloro[1,1′-bis(diphenylphosphino)ferrocene]palladium(II)-dichloromethane adduct (5.0 mg, 0.00613 mmol) and the mixture was purged with nitrogen. 1,4-Dioxane (1 mL) and water (0.5 mL) were added and the mixture was heated overnight at 100° C. LCMS analysis indicated desired product formation and consumption of starting material. The mixture was filtered through a syringe filter and purified by reverse phase HPLC (Gilson, 10-90% MeCN/water+0.1% TFA). The desired fractions were collected and added to a reparatory funnel containing EtOAc and saturated aqueous sodium bicarbonate. The aqueous phase was extracted with EtOAc (3×) and the combined organics were washed with brine, dried over Na₂SO₄ and concentrated to afford 6-{[(3S)-1-(cyclopropylcarbonyl)-3-pyrrolidinyl]methyl}-5-[4-(1H-indol-6-yl)phenyl]-1-methyl-1,5-dihydro-4H-pyrazolo[3,4-d]pyrimidin-4-one (25 mg, 66%) as a tan solid. LCMS (ES⁺) 493.2 [M+H]⁺.

### Example 15

### 6-{[(3S)-1-(cyclopropylcarbonyl)-3-pyrrolidinyl]methyl}-5-[4-(1H-indol-5-yl)phenyl]-1-methyl-1,5-dihydro-4H-pyrazolo[3,4-d]pyrimidin-4-one

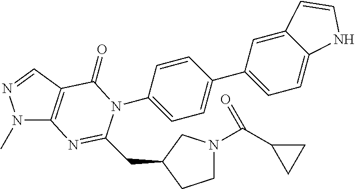 [SMILES: Cn1ncc2c(=O)n(-c3ccc(-c4ccc5[nH]ccc5c4)cc3)c(C[C@@H]3CCN(C(=O)C4CC4)C3)nc21]

The procedure described for the preparation of example 14 (step g) was followed replacing 6-(4,4,5,5-tetramethyl-1,3,2-dioxaborolan-2-yl)-1H-indole with 5-(4,4,5,5-tetramethyl-1,3,2-dioxaborolan-2-yl)-1H-indole which provided the title compound a white solid (10 mg, 43%). LCMS (ES⁺) m/e 493.2 [M+H]⁺.

### Example 16

### 6-[4-(1-benzofuran-5-yl)-2-fluorophenyl]-5-{[(3R)-1-(cyclopropylcarbonyl)-3-pyrrolidinyl]methyl}-1-methyl-1,5-dihydro-4H-pyrazolo[3,4-d]pyrimidin-4-one

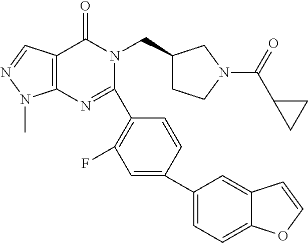 [SMILES: Cn1ncc2c(=O)n(C[C@@H]3CCN(C(=O)C4CC4)C3)c(-c3ccc(-c4ccc5occc5c4)cc3F)nc21]

### a) Ethyl 5-{[(4-bromo-2-fluorophenyl)carbonyl]amino}-1-methyl-1H-pyrazole-4-carboxylate

Ethyl 5-amino-1-methyl-1H-pyrazole-4-carboxylate (1.354 g, 8.00 mmol) was dissolved in toluene (40 mL) in a microwave vial. Hunig's base (2.94 mL, 16.9 mmol) was added and the vial was heated to 60° C. A solution of 4-bromo-2-fluorobenzoyl chloride (2 g, 8.42 mmol) in toluene (2 mL) was added portion wise and the reaction was heated to 90° C. for 0.5 h. The toluene was evaporated and the residue was taken up in ethyl acetate. Crystals which formed after 30 minutes were filtered off and washed with hexane/ethyl acetate 3/1, then hexane. A second crop was obtained by the addition of hexane. The crops were combined to obtain the title compound (3.0 g, 8.10 mmol, 96%). LCMS (ES⁺) m/z 370.0 [M+H]⁺.

Note: the acid chloride was made from the commercially available acid by stirring in dichloromethane with two equivalent of oxalyl chloride and three drops of DMF at room temperature and evaporating the solvent after 3 hr.

### b) 5-{[(4-Bromo-2-fluorophenyl)carbonyl]amino}-1-methyl-1H-pyrazole-4-carboxylic acid

Ethyl 5-{[(4-bromo-2-fluorophenyl)carbonyl]amino}-1-methyl-1H-pyrazole-4-carboxylate (8 g, 21.61 mmol) was dissolved in THF (40 mL) and methanol (100 ml) and 80 mL of 1 N sodium hydroxide was added. After stirring at room temperature for 30 min, LCMS indicated no hydrolysis had taken place. A mixture of 5 g of KOH in 60 mL of water was added and the clear solution was stirred overnight at 40° C. The cooled reaction was adjusted to pH 3 with 6 N HCl. Crystals formed and were filtered off and air dried. The crystals still contained water and were dissolved in THF and the solution dried with sodium sulfate. After filtration, the THF was evaporated and the residue was dissolved in DMF and evaporated to provide the title compound (2.8 g, 8.10 mmol, 37.9%). LCMS (ES⁺) m/z 343.0 [M+H]⁺.

### c) 1,1-Dimethylethyl (3S)-3-({[(5-{[(4-bromo-2-fluorophenyl)carbonyl]amino}-1-methyl-1H-pyrazol-4-yl)carbonyl]amino}methyl)-1-pyrrolidinecarboxylate

5-{[(4-Bromo-2-fluorophenyl)carbonyl]amino}-1-methyl-1H-pyrazole-4-carboxylic acid (1.826 g, 5.34 mmol) was dissolved in dry DMF (20 mL) and 2-chloro-1,3-dimethylimidazolinium chloride (1.082 g, 6.40 mmol) was added and the solution was cooled to 0° C. Hunig's base (1.864 ml, 10.67 mmol) was added and the reaction was warmed to 25° C. for 0.5 h. 1,1-Dimethylethyl (3S)-3-(aminomethyl)-1-pyrrolidinecarboxylate (1.817 g, 9.07 mmol) was added and the reaction was heated to 40° C. overnight. The DMF was removed under reduced pressure and the residue was taken up in dichloromethane/water. The product was extracted into dichloromethane and the organic layer was dried over sodium sulfate. The residue was purified by silica gel chromatography (40 g column, eluting with ethyl acetate) to provide the title compound (2.8 g, 5.30 mmol, 99% yield). LCMS (ES⁺) m/z 524.3 [M+H]⁺.

### d) 5-{[(4-Bromo-2-fluorophenyl)carbonyl]amino}-N-{[(3S)-1-(cyclopropylcarbonyl)-3-pyrrolidinyl]methyl}-1-methyl-1H-pyrazole-4-carboxamide

1,1-Dimethylethyl (3S)-3-({[(5-{[(4-bromo-2-fluorophenyl)carbonyl]amino}-1-methyl-1H-pyrazol-4-yl)carbonyl]amino}methyl)-1-pyrrolidinecarboxylate (3 g, 5.72 mmol) was dissolved in 1,4-dioxane (5 mL). The solution was cooled to 0° C. and 20 mL of 4 N HCl in dioxane was added and the reaction was allowed to warm to 25° C. for 1 h. The dioxane was evaporated under reduced pressure and the residue was dissolved in dichloromethane. Hunig's base (3.0 mL, 17.16 mmol) was added followed by dropwise addition of cyclopropanecarbonyl chloride (0.598 g, 5.72 mmol). After stirring for 1 h at room temperature the solvent and excess Hunig's base were evaporated and the residue was dissolved in dichloromethane and washed with saturated aqueous sodium bicarbonate. The organic layer was dried over sodium sulfate, filtered and concentrated under reduced pressure to give 2.2 g of the crude title compound which was used in the next step without purification. LCMS (ES⁺) m/z 493.0 [M+H]⁺.

### e) 6-(4-Bromo-2-fluorophenyl)-5-{[(3R)-1-(cyclopropylcarbonyl)-3-pyrrolidinyl]methyl}-1-methyl-1,5-dihydro-4H-pyrazolo[3,4-d]pyrimidin-4-one

5-{[(4-Bromo-2-fluorophenyl)carbonyl]amino}-N-{[(3S)-1-(cyclopropylcarbonyl)-3-pyrrolidinyl]methyl}-1-methyl-1H-pyrazole-4-carboxamide (2.0 g, 4.06 mmol) was dissolved in 1,2-dichloroethane (40 mL). The solution was cooled to 0° C. and four equivalents of 1M TiCl₄ in dichloromethane were added. The reaction was heated to 120° C. until it was judged complete by LCMS. The reaction contents were poured onto ice slowly and neutralized to pH 7.5. The mixture was extracted with dichloromethane several times and the extracts were dried over Na₂SO₄, filtered and concentrated. The residue was purified by silica gel chromatography (40 g silica gel column, 50-100% ethyl acetate/hexane gradient) to provide the title compound (375 mg, 0.791 mmol, 19.46%). LCMS (ES) m/z 474.0 [M+H]

### f) 6-[4-(1-Benzofuran-5-yl)-2-fluorophenyl]-5-{[(3R)-1-(cyclopropylcarbonyl)-3-pyrrolidinyl]methyl}-1-methyl-1,5-dihydro-4H-pyrazolo[3,4-d]pyrimidin-4-one

6-(4-Bromo-2-fluorophenyl)-5-{[(3R)-1-(cyclopropylcarbonyl)-3-pyrrolidinyl]methyl}-1-methyl-1,5-dihydro-4H-pyrazolo[3,4-d]pyrimidin-4-one (75 mg, 0.158 mmol) and 5-(4,4,5,5-tetramethyl-1,3,2-dioxaborolan-2-yl)-1-benzofuran (38.6 mg, 0.158 m mol) were dissolved in 1,4-dioxane (2 mL) in a sealable reaction vial and PdCl₂(dppf)-CH₂Cl₂ adduct (6.46 mg, 7.91 μmol) and 200 ul of 2N K₂CO₃ were added. The vial was capped, flushed with nitrogen and heated to 100° C. for 1 h. The reaction mixture was adjusted to pH 8 with 1 N HCl. The dioxane was evaporated and the residue was partitioned between water and dichloromethane. DMSO was added to the dichloromethane layer and the dichloromethane layer was evaporated. Purification on a reverse phase HPLC column using a 10 minute gradient and eluting with 10-90% acetonitrile/water containing 0.1% TFA provided the title compound (26 mg, 0.051 mmol, 32%). LCMS (ES⁺) m/z 511.0 [M+H]⁺.

### Example 17

### 5-{[(3R)-1-(cyclopropylcarbonyl)-3-pyrrolidinyl]methyl}-6-(3,4′-difluoro-4-biphenylyl)-1-methyl-1,5-dihydro-4H-pyrazolo[3,4-d]pyrimidin-4-one

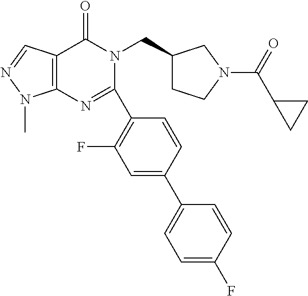 [SMILES: Cn1ncc2c(=O)n(C[C@@H]3CCN(C(=O)C4CC4)C3)c(-c3ccc(-c4ccc(F)cc4)cc3F)nc21]

Following the procedure described in Example 16 with (4-fluorophenyl)boronic acid provided the title compound (30.1 mg, 0.061 mmol, 38.5%). LCMS (ES⁺) m/z 490.5 [M+H]⁺.

### Example 18

### 5-{[(3R)-1-(cyclopropylcarbonyl)-3-pyrrolidinyl]methyl}-6-[2-fluoro-4-(1H-indol-5-yl)phenyl]-1-methyl-1,5-dihydro-4H-pyrazolo[3,4-d]pyrimidin-4-one

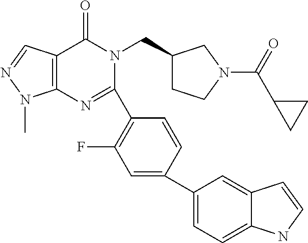 [SMILES: Cn1ncc2c(=O)n(C[C@@H]3CCN(C(=O)C4CC4)C3)c(-c3ccc(-c4ccc5[nH]ccc5c4)cc3F)nc21]

Following the procedure described in Example 16 with 1H-indol-5-ylboronic acid provided the title compound (27.8 mg, 0.054 mmol, 26.4%). LCMS (ES⁺) m/z 511.5 [M+H]⁺.

### Example 19

### 5-{[(3R)-1-(cyclopropylcarbonyl)-3-pyrrolidinyl]methyl}-6-[2-fluoro-4-(1H-indol-6-yl)phenyl]-1-methyl-1,5-dihydro-4H-pyrazolo[3,4-d]pyrimidin-4-one

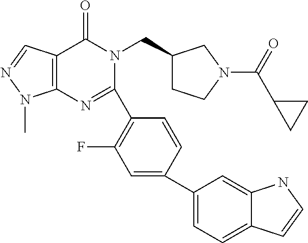 [SMILES: Cn1ncc2c(=O)n(C[C@@H]3CCN(C(=O)C4CC4)C3)c(-c3ccc(-c4ccc5cc[nH]c5c4)cc3F)nc21]

Following the procedure described in Example 16 with 1H-indol-6-ylboronic acid provided the title compound (44.5 mg, 0.086 mmol, 42.2%). LCMS (ES⁺) m/z 511.5 [M+H]⁺.

### Example 20

### 2-[4-(1-benzofuran-5-yl)phenyl]-3-{[(3R)-1-(cyclopropylcarbonyl)-3-pyrrolidinyl]methyl}-4(3H)-quinazolinone

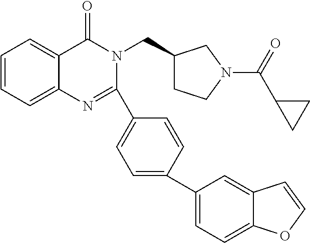 [SMILES: O=C(C1CC1)N1CC[C@@H](Cn2c(-c3ccc(-c4ccc5occc5c4)cc3)nc3ccccc3c2=O)C1]

### a) ethyl 2-{[(4-bromophenyl)carbonyl]amino} benzoate

Ethyl 2-aminobenzoate (12 g, 72.6 mmol) and DMAP (0.177 g, 1.453 mmol) were added to a mixture of dichloromethane (75 mL), pyridine (20 mL) and 4 Å molecular sieves. 4-Bromobenzoyl chloride (15.94 g, 72.6 mmol) dissolved in 30 mL of CH₂Cl₂ was added dropwise over 2 h and the reaction was stirred at room temperature for 18 h. The reaction mixture was diluted with a large volume of ethyl acetate and washed with water containing 1 N hydrochloric acid until slightly acidic (pH 3.5). The ethyl acetate solution was dried with sodium sulfate. Partial evaporation of the solvent gave several crops of product which still contained some starting material. This material was taken up in dichloromethane (staring material was not soluble), filtered and the filtrate was concentrated under reduced pressure to give ethyl 2-{[(4-bromophenyl)carbonyl]amino}benzoate (15 g, 43.1 mmol, 59.3% yield). LCMS (ES⁺) m/z 348.0 [M+H]⁺.

### b) 2-{[(4-bromophenyl)carbonyl]amino}benzoic acid

To ethyl 2-{[(4-bromophenyl)carbonyl]amino}benzoate (3 g, 8.62 mmol) in methanol (30 mL) was added 26 ml of 1 N sodium hydroxide and the reaction was heated at 55° C. overnight. The reaction mixture was acidified with 1 N hydrochloric acid and concentrated until a solid formed. The solid was filtered off and washed with water. The solid was taken up in ethyl acetate, the water separated and the organic solution was dried with sodium sulfate and concentrated under reduced pressure to give 2-{[(4-bromophenyl)carbonyl]amino}benzoic acid (2.57 g, 8.03 mmol, 93% yield), as a white solid. LCMS (ES⁺) m/z 320.0 [M+H]⁺.

### c) 2-(4-bromophenyl)-4H-3,1-benzoxazin-4-one

2-{[(4-bromophenyl)carbonyl]amino}benzoic acid (2.57 g, 8.03 mmol) and acetic anhydride (45.4 ml, 482 mmol) were added to a 150 mL flask and the contents were heated in an oil bath at 140° C. for 30 min. The acetic anhydride was removed under reduced pressure at 70° C. The solid obtained was triturated with hexane and collected by filtration to provide 2-(4-bromophenyl)-4H-3,1-benzoxazin-4-one (2.2 g, 7.28 mmol, 91% yield). LCMS (ES⁺) m/z 302.2 [M+H]⁺.

### d) 2-{[(4-bromophenyl)carbonyl]amino}-N-{[(3S)-1-(cyclopropylcarbonyl)-3-pyrrolidinyl]methyl}benzamide

2-(4-Bromophenyl)-4H-3,1-benzoxazin-4-one (3.5 g, 11.58 mmol) and 1-[(3S)-1-(cyclopropylcarbonyl)-3-pyrrolidinyl]methanamine (2.14 g, 12.74 mmol, 95% ee) were added to 1,4-dioxane (42 mL) and the solution was heated under nitrogen to 60° C. for 2 h. LCMS analysis indicated complete consumption of starting material. The product crystallized out as a white solid which was collected by filtration and washed with dioxane and hexane to provide 2-[(4-bromophenyl)carbonyl]amino}-N-{[(3S)-1-(cyclopropylcarbonyl)-3-pyrrolidinyl]methyl}benzamide (5 g, 9.78 mmol, 84% yield) as a white solid. LCMS (ES⁺) m/z 470.0 [M+H]⁺.

### e) 2-(4-bromophenyl)-3-{[(3R)-1-(cyclopropylcarbonyl)-3-pyrrolidinyl]methyl}-4(3H)-quinazolinone

2-{[(4-Bromophenyl)carbonyl]amino}-N-{[(3S)-1-(cyclopropylcarbonyl)-3-pyrrolidinyl]methyl}benzamide (5.5 g, 11.69 mmol) and sodium hydroxide (0.180 g, 4.50 mmol) were added to ethylene glycol (196 mL) and the solution was heated under nitrogen to 100° C. for 2 h. After 2 h, water was added and the mixture was stirred and neutralized with 1 N HCl to pH of 6-7. The white solid containing the product was filtered off and air dried. The crude residue was dry packed on silica gel and eluted off a 40 g silica gel column with 20-100% ethyl acetate/hexane to provide 2-(4-bromophenyl)-3-{[(3R)-1-(cyclopropylcarbonyl)-3-pyrrolidinyl]methyl}-4(3H)-quinazolinone (1.73 g, 3.75 mmol, 32.1% yield; 670 mg of starting material was also recovered). LCMS (ES⁺) m/z 453.0 [M+H]⁺.

### f) 2-[4-(1-benzofuran-5-yl)phenyl]-3-{[(3R)-1-(cyclopropylcarbonyl)-3-pyrrolidinyl]methyl}-4(3H)-quinazolinone

2-(4-Bromophenyl)-3-{[(3R)-1-(cyclopropylcarbonyl)-3-pyrrolidinyl]methyl}-4(3H)-quinazolinone (100 mg, 0.221 mmol), 5-(4,4,5,5-tetramethyl-1,3,2-dioxaborolan-2-yl)-1-benzofuran (81 mg, 0.332 mmol) and Pd₂(dppf)-CH₂Cl₂ adduct (9.0 mg, 0.011 mmol) were dissolved in dioxane (2 mL) in a microwave vial and 2M aqueous potassium carbonate (0.25 mL) was added. The microwave vial was flushed with nitrogen, capped and heated to 100° C. for 1 h. The dioxane was removed under reduced pressure and the residue was diluted with ethyl acetate, washed with water, dried over sodium sulfate, filtered and concentrated under reduced pressure. The residue was purified by silica gel chromatography using a 4 g silica column (eluting with 10-100% ethyl acetate/hexane) to provide 2-[4-(1-benzofuran-5-yl)phenyl]-3-{[(3R)-1-(cyclopropylcarbonyl)-3-pyrrolidinyl]methyl}-4(3H)-quinazolinone (84 mg, 0.160 mmol, 72.2% yield). LCMS (ES⁺) m/z 490.2 [M+H]⁺.

### Example 21

### 3-{[(3R)-1-(cyclopropylcarbonyl)-3-pyrrolidinyl]methyl}-2-[4-(1H-indol-6-yl)phenyl]-4(3H)-quinazolinone

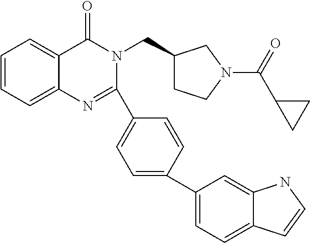 [SMILES: O=C(C1CC1)N1CC[C@@H](Cn2c(-c3ccc(-c4ccc5cc[nH]c5c4)cc3)nc3ccccc3c2=O)C1]

Following the procedure described in Example 20 (step f) with 1H-indol-6-ylboronic acid provided the title product (49 mg, 0.098 mmol, 44.5%) as a solid. LCMS (ES⁺) m/z 489.4 [M+H]⁺.

### Example 22

### 3-{[(3R)-1-(cyclopropylcarbonyl)-3-pyrrolidinyl]methyl}-2-[4-(1H-indol-5-yl)phenyl]-4(3H)-quinazolinone

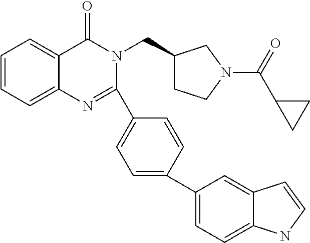 [SMILES: O=C(C1CC1)N1CC[C@@H](Cn2c(-c3ccc(-c4ccc5[nH]ccc5c4)cc3)nc3ccccc3c2=O)C1]

Following the procedure described in Example 20 (step f) with 1H-indol-5-ylboronic acid provided the title product (49 mg, 0.098 mmol, 44.5%) as a solid. LCMS (ES⁺) m/z 489.4 [M+H]⁺.

### Example 23

### 3-{[(3R)-1-(cyclopropylcarbonyl)-3-pyrrolidinyl]methyl}-2-[4′-(methyloxy)-4-biphenylyl]-4(3H)-quinazolinone

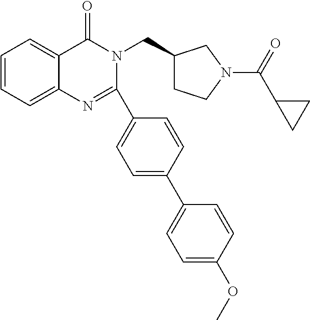 [SMILES: COc1ccc(-c2ccc(-c3nc4ccccc4c(=O)n3C[C@@H]3CCN(C(=O)C4CC4)C3)cc2)cc1]

Following the procedure described in Example 20 (step f) with 4-(methyloxy)phenyl]boronic acid provided the title product (49 mg, 0.1 mmol, 45%) as a solid. LCMS (ES⁺) m/z 480.4 [M+H]⁺.

### Example 24

### 2-[2-chloro-4(methoxy)-4-bipheny]-3-{[(3R)-1-(cyclopropylcarbonyl)-3-pyrrolidinyl]methyl}-4(3H)-quinazolinone

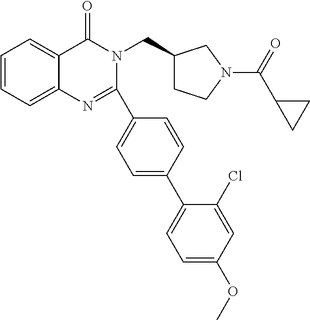 [SMILES: COc1ccc(-c2ccc(-c3nc4ccccc4c(=O)n3C[C@@H]3CCN(C(=O)C4CC4)C3)cc2)c(Cl)c1]

Following the procedure described in Example 20 (step f) with [2-chloro-4-(methyloxy)phenyl]boronic acid provided the title product (100 mg, 0.191 mmol, 86%) as a solid. LCMS (ES⁺) m/z 514.3.4 [M+H]⁺.

### Example 25

### 2-[4-(1-benzofuran-D-phenyl]-3-{[3R)-1-(cyclopropylcarbonyl)-3-pyrrolidinyl]methyl}-6-methyl-4(3H)-pyrimidinone

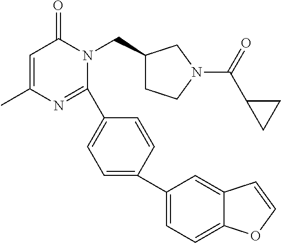 [SMILES: Cc1cc(=O)n(C[C@@H]2CCN(C(=O)C3CC3)C2)c(-c2ccc(-c3ccc4occc4c3)cc2)n1]

### a) N-{[(3S)-1-(cyclopropylcarbonyl)-3-pyrrolidinyl]methyl}-3-oxobutanamide

4-Methylidene-2-oxetanone (1.0 g, 11.89 mmol) was dissolved in diethyl ether (10 mL) and cooled to 0° C. To this solution was added dropwise a solution of 1-[(3S)-1-(cyclopropylcarbonyl)-3-pyrrolidinyl]methanamine (2.00 g, 11.89 mmol, prepared as shown below) in chloroform (10 mL). The solution was allowed to warm to room temperature then refluxed for 3 h. The reaction mixture was washed with 1 N HCl and water. The aqueous layer was neutralized and extracted two times with dichloromethane. The organics were combined, dried with sodium sulfate, filtered and concentrated under reduced pressure to provide N-{[(3S)-1-(cyclopropylcarbonyl)-3-pyrrolidinyl]methyl}-3-oxobutanamide (2.0 g, 6.74 mmol, 56.6% yield). LCMS (ES⁺) m/z 253.2 [M+H]⁺.

### Preparation of 1-[(3S)-1-(cyclopropylcarbonyl)-3-pyrrolidinyl]methanamine

### i) (3R)-1-(cyclopropylcarbonyl)-3-pyrrolidinecarbonitrile

A solution of 1,1-dimethylethyl (3R)-3-cyano-1-pyrrolidinecarboxylate (27 g, 138 mmol) in EtOH (200 mL) was treated with 4 M HCl in 1,4-dioxane (120 mL, 480 mmol) and stirred for 2 h. The mixture was evaporated to an oil, then azeotroped with EtOH and CHCl₃. The residue was taken up in CHCl₃ (300 mL) and DIPEA (71.9 mL, 413 mmol) and cooled over an ice bath. The mixture was treated with cyclopropanecarbonyl chloride (14.98 mL, 165 mmol) in CHCl₃ (100 mL), the ice bath was removed, and the mixture was stirred for 2 h. The mixture was washed with 1 M HCl and brine, dried over Na₂SO₄, and evaporated. Flash chromatography (CH₂Cl₂-5% MeOH in CH₂Cl₂) afforded the title compound (22 g, 97%). LCMS (ES⁺) 165 [M+H]⁺.

### ii) 1-[(3S)-1-(cyclopropylcarbonyl)-3-pyrrolidinyl]methanamine

A solution of (3R)-1-(cyclopropylcarbonyl)-3-pyrrolidinecarbonitrile (12 g, 73.1 mmol) in EtOH (900 mL) and ammonia solution (45 mL, 73.1 mmol) was divided between 3×500 mL Parr flasks, flushed with N₂, and raney nickel was added. The mixtures were placed on Parr shakers, flushed several times with N₂, and shaken under a hydrogen atmosphere at 60 psi for 3 h. The contents of each flask were flushed with N₂ and filtered through Celite® under a N₂ atmosphere, keeping the catalyst wet, washing through with a little EtOH then immediately dousing with water. The solvent was evaporated to give a clear oil of the desired compound (11.1 g, 90%; 84% pure and ˜88% ee). Purification by preparative chiral HPLC (Chiralpak AD 20 μL, heptane:EtOH:isopropylamine 75:25:0.1; UV 220 nm) afforded the title compound (99.4% ee). LCMS (ES⁺) 169 [M+H]⁺.

### b) 2-(4-bromophenyl)-3-{[(3R)-1-(cyclopropylcarbonyl)-3-pyrrolidinyl]methyl}-6-methyl-4(3H)-pyrimidinone

N-{[(3S)-1 (Cyclopropylcarbonyl)-3-pyrrolidinyl]methyl}-3-oxobutanamide (500 mg, 1.982 mmol) and 4-bromobenzamide (476 mg, 2.38 mmol) were added to a sealable reaction vial (oven dried). Titanium (IV) isopropoxide (2.3 mL, 7.93 mmol) was added and the vial was capped under nitrogen. The reaction mixture was heated at 150° C. for 24 h, cooled to room temperature and toluene (6 mL) and 2N HO (6 mL) were added and the reaction was stirred for 2 h at room temperature. The layers were separated and the aqueous layer was extracted with toluene and dichloromethane. The combined organic extracts were washed with brine, dried over sodium sulfate and evaporated to obtain 550 mg of 2-(4-bromophenyl)-3-{[(3R)-1-(cyclopropylcarbonyl)-3-pyrrolidinyl]methyl}-6-methyl-4(3H)-pyrimidinone. LCMS (ES⁺) m/z 418.2 [M+H]⁺.

### c) 2-[4-(1-benzofuran-5-yl)phenyl]-3-{[(3R)-1-(cyclopropylcarbonyl)-3-pyrrolidinyl]methyl}-6-methyl-4(3H)-pyrimidinone

2-(4-Bromophenyl)-3-{[(3R)-1-(cyclopropylcarbonyl)-3-pyrrolidinyl]methyl}-6-methyl-4(3H)-pyrimidinone (120 mg, 0.288 mmol) and 5-(4,4,5,5-tetramethyl-1,3,2-dioxaborolan-2-yl)-1-benzofuran (77 mg, 0.317 mmol) were dissolved in 1,4-dioxane (2 mL) in a sealable reaction vial. PdCl₂(dppf)-CH₂Cl₂ adduct (11.8 mg, 0.014 mmol) and 2N K₂CO₃ (0.4 mL) were added. The vial was capped, flushed with nitrogen and heated to 100° C. for 1 h. The dioxane was evaporated under reduced pressure and the residue was taken up in ethyl acetate and the aqueous layer was adjusted to pH 7 with 1N HCl. The ethyl acetate solution was washed with water, dried over sodium sulfate, filtered and concentrated under reduced pressure. The residue was purified on silica (2 g) eluting with 30-100% ethyl acetate in hexane and then 0.5% methanol in ethyl acetate to give 2-[4-(1-benzofuran-5-yl)phenyl]-3-{[(3R)-1-(cyclopropylcarbonyl)-3-pyrrolidinyl]methyl}-6-methyl-4(3H)-pyrimidinone (101 mg, 0.209 mmol, 72.6% yield). LCMS (ES⁺) m/z 454.4 [M+H]⁺.

### Example 26

### 3-{[(3R)-1-(cyclopropylcarbonyl)-3-pyrrolidinyl]methyl}-2-[4-(1H-indol-5-yl)phenyl-6-methyl-4(3H)-pyrimidinone

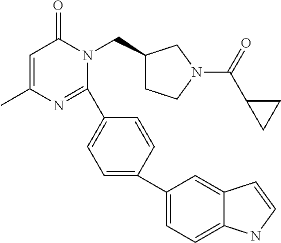 [SMILES: Cc1cc(=O)n(C[C@@H]2CCN(C(=O)C3CC3)C2)c(-c2ccc(-c3ccc4[nH]ccc4c3)cc2)n1]

Following the procedure described in Example 25 (step c) with 5-(4,4,5,5-tetramethyl-1,3,2-dioxaborolan-2-yl)-1H-indole provided the title product (90 mg, 0.199 mmol, 64%). LCMS (ES⁺) m/z 453.1 [M+H]⁺.

### Example 27

### 3-{[(3R)-1-(cyclopropylcarbonyl)-3-pyrrolidinyl]methyl}-2-[4-(1H-indol-6-yl)phenyl-6-methyl-4(3H)-pyrimidinone

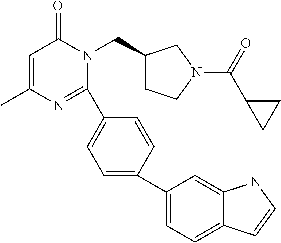 [SMILES: Cc1cc(=O)n(C[C@@H]2CCN(C(=O)C3CC3)C2)c(-c2ccc(-c3ccc4cc[nH]c4c3)cc2)n1]

Following the procedure described in Example 25 (step c) with 1H-indol-6-ylboronic acid provided the title product (32 mg, 0.069 mmol, 26%). LCMS (ES⁺) m/z 453.4 [M+H]⁺.

### Example 28

### 4-({[(3R)-1-(cyclopropylcarbonyl)-3-pyrrolidinyl]methyl}oxy)-6-methyl-2-[4′-(methyloxy)-4-biphenylyl]pyrimidine

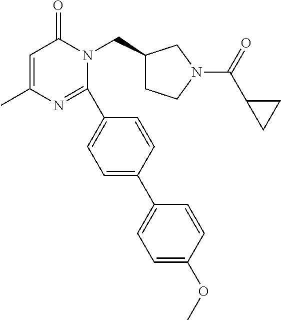 [SMILES: COc1ccc(-c2ccc(-c3nc(C)cc(=O)n3C[C@@H]3CCN(C(=O)C4CC4)C3)cc2)cc1]

Following the procedure described in Example 25 (step c) with [4-(methyloxy)phenyl]boronic acid provided the title product (50 mg, 0.106 mmol, 43%). LCMS (ES⁺) m/z 444.1 [M+H]⁺.

### Example 29

### 2-[2′-chloro-4′-(methyloxy)-4-biphenylyl]-3-{[(3R)-1-(cyclopropylcarbonyl)-3-pyrrolidinyl]methyl}-6-methyl-4(3H)-pyrimidinone

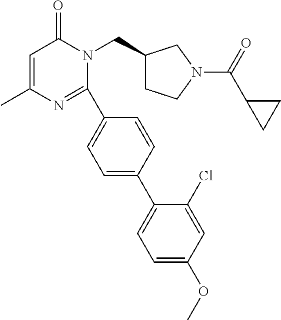 [SMILES: COc1ccc(-c2ccc(-c3nc(C)cc(=O)n3C[C@@H]3CCN(C(=O)C4CC4)C3)cc2)c(Cl)c1]

Following the procedure described in Example 25 (step c) with [2-chloro-4-(methyloxy)phenyl]boronic acid provided the title product (50 mg, 0.102 mmol, 40%). LCMS (ES⁺) m/z 478.11 [M+H]⁺.

### Example 30

N-[4′-(1-{[(3R)-1-(cyclopropylcarbonyl)-3-pyrrolidinyl]methyl}-4-methyl-6-oxo-1,6-dihydro-2-pyrimidinyl)-3-biphenylyl]-N,N-dimethylsulfamide

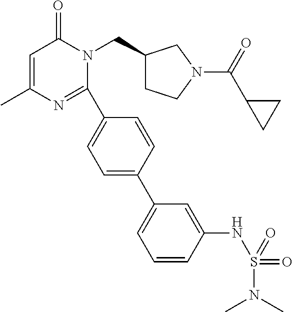 [SMILES: Cc1cc(=O)n(C[C@@H]2CCN(C(=O)C3CC3)C2)c(-c2ccc(-c3cccc(NS(=O)(=O)N(C)C)c3)cc2)n1]

Following the procedure described in Example 25 (step c) with (3-{[(dimethylamino)sulfonyl]amino}phenyl)boronic acid provided the title product (79 mg, 0,146 mmol, 49%), LCMS (ES⁺) m/z 536.0 [M+H]⁺.

### Example 31

### 3-{[(3R)-1-(cyclopropylcarbonyl)-3-pyrrolidinyl]methyl}-2-(4′-fluoro-4-biphenylyl)-6-methyl-4(3H)-pyrimidinone

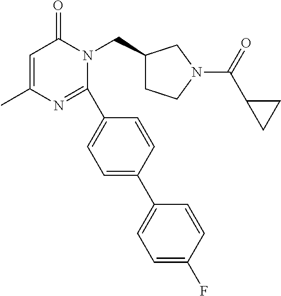 [SMILES: Cc1cc(=O)n(C[C@@H]2CCN(C(=O)C3CC3)C2)c(-c2ccc(-c3ccc(F)cc3)cc2)n1]

Following the procedure described in Example 25 (step c) with (4-fluorophenyl)boronic acid and purification using reverse phase HPLC (10 minute gradient of 10-90% acetonitrile/water containing 0.1% TFA) provided the title product (65 mg, 0.148 mmol, 77%), LCMS (ES⁺) m/z 432.0 [M+H]⁺.

### Example 32

### 2-[4-(1-benzofuran-5-yl)-2-fluorophenyl]-3-{[(3R)-1-(cyclopropylcarbonyl)-3-pyrrolidinyl]methyl}-6-methyl-4(3H)-pyrimidinone

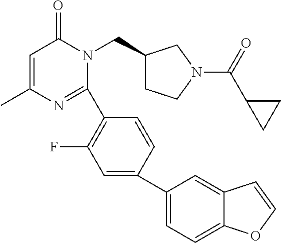 [SMILES: Cc1cc(=O)n(C[C@@H]2CCN(C(=O)C3CC3)C2)c(-c2ccc(-c3ccc4occc4c3)cc2F)n1]

### a) 2-(4-bromo-2-fluorophenyl)-3-{[(3R)-1-(cyclopropylcarbonyl)-3-pyrrolidinyl]methyl}-6-methyl-4(3H)-pyrimidinone

Following the procedure described in Example 25 (step b) replacing 4-bromobenzamide with 4-bromo-2-fluorobenzamide provided the title product (762 mg, 1.5 mmol, 27%). LCMS (ES⁺) m/z 435.2 [M+H]⁺.

### b) 2-[4-(1-benzofuran-5-yl)-2-fluorophenyl]-3-{[(3R)-1-(cyclopropylcarbonyl)-3-pyrrolidinyl]methyl}-6-methyl-4(3H)-pyrimidinone

Following the procedure described in Example 25 (step c) with 5-(4,4,5,5-tetramethyl-1,3,2-dioxaborolan-2-yl)-1-benzofuran and purification using reverse phase HPLC (10 minute gradient of 10-90% acetonitrile/water containing 0.1% TFA) provided the title product (66 mg, 0.133 mmol, 58%). LCMS (ES⁺) m/z 472.4.0 [M+H]⁺.

### Example 33

### 3-{[(3R)-1-(cyclopropylcarbonyl)-3-pyrrolidinyl]methyl}-2-[2-fluoro-4-(1H-indol-5-yl)phenyl]-6-methyl-4(3H)-pyrimidinone

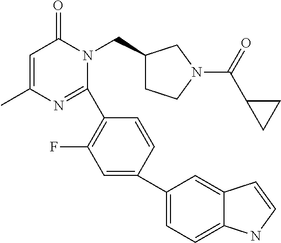 [SMILES: Cc1cc(=O)n(C[C@@H]2CCN(C(=O)C3CC3)C2)c(-c2ccc(-c3ccc4[nH]ccc4c3)cc2F)n1]

Following the procedure described in Example 25 (step c) with 544,4,5,5-tetramethyl-1,3,2-dioxaborolan-2-yl)-1H-indole and purification using reverse phase HPLC (10 minute gradient of 10-90% acetonitrile/water containing 0.1% TFA) provided the title product (36 mg, 0.073 mmol, 58%). LCMS (ES⁺) m/z. 471.4 [M+H]⁺.

### Example 34

### 3-{[(3R)-1-(cyclopropylcarbonyl)-3-pyrrolidinyl]methyl}-2-[2-fluoro-4-(1H-indol-6-yl)phenyl]-6-methyl-4(3H)-pyrimidinone

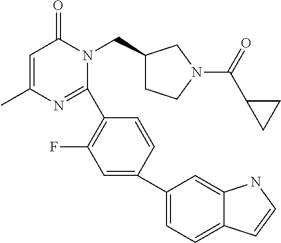 [SMILES: Cc1cc(=O)n(C[C@@H]2CCN(C(=O)C3CC3)C2)c(-c2ccc(-c3ccc4cc[nH]c4c3)cc2F)n1]

Following the procedure described in Example 25 (step c) with 1H-indol-6-ylboronic acid and purification using reverse phase HPLC (10 minute gradient of 10-90% acetonitrile/water containing 0.1% TFA) provided the title product (50 mg, 0.104 mmol, 45%). LCMS (ES⁺) m/z 471.4 [M+H]⁺.

### Example 35

### 3-{[(3R)-1-(cyclopropylcarbonyl)-3-pyrrolidinyl]methyl}-2-(3,4′-difluoro-4-biphenylyl)-6-methyl-4(3H)-pyrimidinone

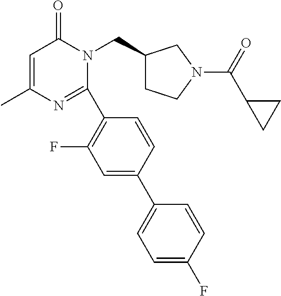 [SMILES: Cc1cc(=O)n(C[C@@H]2CCN(C(=O)C3CC3)C2)c(-c2ccc(-c3ccc(F)cc3)cc2F)n1]

Following the procedure described in Example 25 (step c) with (4-fluorophenyl)boronic acid and purification using reverse phase HPLC provided the title product (14 mg, 0.030 mmol, 13%). LCMS (ES⁺) m/z, 450.4 [M+H]⁺.

### Example 36

### 3-{[(3R)-1-(cyclopropylcarbonyl)-3-pyrrolidinyl]methyl}-5,6-dimethyl-2-[4′-(methyloxy)-4-biphenylyl]-4(3H)-pyrimidinone

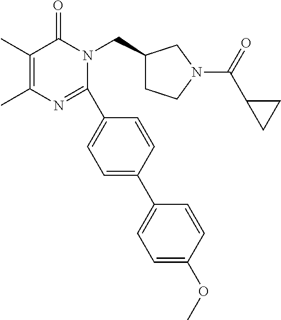 [SMILES: COc1ccc(-c2ccc(-c3nc(C)c(C)c(=O)n3C[C@@H]3CCN(C(=O)C4CC4)C3)cc2)cc1]

### a) ethyl 2-(2-methyl-1,3-dioxolan-2-yl)propanoate

Ethyl 2-methyl-3-oxohutanoate (14 g, 97 mmol), 1,2-ethanediol (6.33 g, 102 mmol), p-toluenesulfonic acid (1.85 g, 9.71 mmol) and 3 Å molecular sieves were added to 150 mL of toluene. The mixture was refluxed, with stirring, for 3 days, The reaction mixture was allowed to cool to room temperature and the solvent was evaporated. The crude product was dissolved in ethyl acetate, washed with saturated bicarbonate solution, dried over sodium sulfate and filtered. Evaporation of the ethyl acetate gave ethyl 2-(2-methyl-1,3-dioxolan-2-yl)propanoate (10 g, 53.1 mmol, 54.7% yield). LCMS (ES⁺) m/z 189.2 [M+H]⁺.

### b) 2-(2-methyl-1,3-dioxolan-2-yl)propanoic acid

Ethyl 2-(2-methyl-1,3-dioxolan-2-yl)propanoate (9.41 g, 50 mmol) was disolved in methanol (75 mL) and water (75 mL) containing potassium hydroxide (8.42 g, 150 mmol). The mixture was heated to 65° C. and stirred for 18 h. The methanol was evaporated under reduced pressure and the water solution extracted with ethyl acetate to remove any remaining starting material. The aqueous was acidified to pH 2 with 6N HCl and extracted with ethyl acetate two times. Evaporation of the ethyl acetate provided 2-(2-methyl-1,3-dioxotan-2-yl)propanoic acid (4.5 g, 28.1 mmol, 56.2% yield). LCMS (ES⁺) m/z 161.1 [M+H]⁺.

### c) 2-(2-methyl-1,3-dioxolan-2-yl)propanoyl chloride

2-(2-Methyl-1,3-dioxolan-2-yl)propanoic acid (2.0 g, 12.49 mmol) was dissolved in 20 mL of dichloromethane. The solution was cooled to 0° C. in an ice bath and oxalyl chloride (2.51 mL, 28.7 mmol) was added and the reaction was stirred at 25° C. for 2 h. The solvent was evaporated under reduced pressure to provide 2-(2-methyl-1,3-dioxolan-2-yl)propanoyl chloride (22 g, 12.2 mmol, 99% yield). LCMS (ES⁺) in/Z 179.2 [M+H]⁺.

### d) N-{[(3S)-1-(cyclopropylcarbonyl)-3-pyrrolidinyl]methyl}-2-(2-methyl-1,3-dioxolan-2-yl)propanamide

{[(3S)-1-(Cyclopropylcarbonyl)-3-pyrrolidinyl]methyl} amine (942 mg, 5.60 tumor) was dissolved in 15 mL of dichloromethane and cooled to 0° C. Pyridine (2.5 mL) was slowly added followed by 2-(2-methyl-1,3-dioxolan-2-yl)propanoyl chloride (1.0 g, 5.60 mmol). The reaction mixture was stirred at 25° C. for 18 h. The solvent was removed under reduced pressure and the residue was diluted with saturated aqueous sodium bicarbonate and dichloromethane (30 mL). The aqueous layer was extracted exhaustively with dichloromethane. The combined extracts were concentrated under reduced pressure and the residue was purified on a 12 g silica gel column eluting with 0-15% isopropanol in dichloromethane to provide N-{[(3S)-1-(cyclopropylcarbonyl)-3-pyrrolidinyl]methyl}-2-(2-methyl-1,3-dioxolan-2-yl)propanamide (513 mg, 1.653 mmol, 29.5% yield). LCMS (ES⁺) m/z 311.1 [M+H]⁺.

### e) N-{[(3S)-1-(cyclopropylcarbonyl)-3-pyrrolidinyl]methyl}-2-methyl-3-oxobutanamide

N-{[(3S)-1-(Cyclopropylcarbonyl)-3-pyrrolidinyl]methyl}-2-(2-methyl-1,3-dioxolan-2-yl)propanamide (500 mg, 1.611 mmol) was dissolved in acetone (15 mL) and cooled to 0° C. p-Toluenesulfonic acid (30.6 mg, 0.161 minor) dissolved in 10 mL of water was added and the mixture was heated at 95° C. for 3 h. The solvent was removed under reduced pressure and the residue was dissolved in dichloromethane, washed with saturated aqueous sodium bicarbonate solution, dried over sodium sulfate, filtered and concentrated under reduced pressure to provide N-{[(3S)-1-(cyclopropylcarbonyl)-3-pyrrolidinyl]methyl}-2-methyl-3-oxobutanamide (320 mg, 1.20 mmol, 74.6% yield). LCMS (ES⁺) m/z 267.1 [M+H]+.

### f) 2-(4-bromo-1,5-cyclohexadien-1-yl)-3-{[(3S)-1-(cyclopropylcarbonyl)-3-pyrrolidinyl]methyl}-5,6-dimethyl-4(3H)-pyrimidinone

N-{[(3S)-1-(cyclopropylcarbonyl)-3-pyrrolidinyl]methyl}-2-methyl-3-oxobutanamide (320 mg, 1.20 mmol) and 4-bromobenzamide (288 mg, 1.442 mmol) were added to titanium(IV) isopropoxide (1.41 ml, 4.81 mmol) in a sealable reaction vial. The capped vial was heated to 150° C. for 24 h. The reaction mixture was allowed to cool to room temperature, diluted with toluene (4 mL) and 2N HCl (4 mL) and stirred at room temperature for 2 h. The layers were separated and the aqueous layer was extracted with toluene several times and then with dichloromethane. The organics were combined, washed with brine, dried over sodium sulfate and filtered. The solvent was evaporated under reduced pressure and the residue was purified by silica gel chromatography on a 12 g column eluting with 0-20% isopropanol/dichloromethane to provide 2-(4-bromo-1,5-cyclohexadien-1-yl)-3-{[(3,S)-1-(cyclopropylcarbonyl)-3-pyrrolidinyl]methyl}-5,6-dimethyl-4(3H)-pyrimidinone (370 mg, 0.757 mmol, 63%). LCMS (ES⁺) m/z 431.3 [M+H]⁺.

### g) 3-{[(3R)-1-(cyclopropylcarbonyl)-3-pyrrolidinyl]methyl}-5,6-dimethyl-2-[4′-(methyloxy)-4-biphenylyl]-4(3H)-pyrimidinone

2-(4-Bromophenyl)-3-{[(3R)-1-(cyclopropylcarbonyl)-3-pyrrolidinyl]methyl}-5,6-dimethyl-4(3H)-pyrimidinone (80 mg, 0.186 mmol) and [4-(methyloxy)phenyl]boronic acid (28.2 mg, 0.186 mmol) were dissolved in dioxane (2 mL). PdCl₂(dppf)-CH₂Cl₂ adduct (7.59 mg, 9.30 μmol) and 2M K₂CO₃ (0.3 mL) were added and the vial was capped, flushed with nitrogen and heated to 100° C. for 1 h. The dioxane was evaporated and the crude residue was dissolved in ethyl acetate, washed with water, dried over sodium sulfate, filtered and concentrated under reduced pressure. The residue was purified by silica gel chromatography (eluting with 30-100% ethyl acetate/hexane then 0.5% methanol/ethyl acetate) to give 87 mg of product which was further purified by reverse phase HPLC (10 minute gradient 10-90% acetonitrile/water containing 0.1% TFA) to provide 3-{[(3R)-1-(cyclopropylcarbonyl)-3-pyrrolidinyl]methyl}-5,6-dimethyl-2-[4′-(methyloxy)-4-biphenylyl]-4(3H)-pyrimidinone (17.2 mg, 0.036 mmol, 19% yield). LCMS (ES⁺) m/z 458.3 [M+H]⁺.

### Example 37

### 2-[4-(1-benzofuran-5-yl)phenyl]-3-{[(3R)-1-(cyclopropylcarbonyl)-3-pyrrolidinyl]methyl}-5,6-dimethyl-4(3H)-pyrimidinone

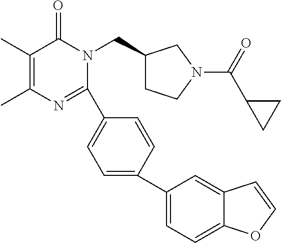 [SMILES: Cc1nc(-c2ccc(-c3ccc4occc4c3)cc2)n(C[C@@H]2CCN(C(=O)C3CC3)C2)c(=O)c1C]

Following the procedure described in Example 36 (step g) with 5-(4,4,5,5-tetramethyl-1,3,2-dioxaborolan-2-yl)-1-benzofuran provided the title product (18.6 mg, 0.038 mmol, 20%). LCMS (ES⁺) m/z 468.4 [M+H]⁺.

### Example 38

### 3-{[(3R)-1-(cyclopropylcarbonyl)-3-pyrrolidinyl]methyl}-2-[4-(1H-indol-6-yl)phenyl]-5,6-dimethyl-4(3H)-pyrimidinone

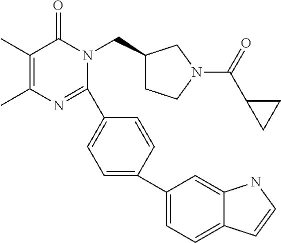 [SMILES: Cc1nc(-c2ccc(-c3ccc4cc[nH]c4c3)cc2)n(C[C@@H]2CCN(C(=O)C3CC3)C2)c(=O)c1C]

Following the procedure described in Example 36 (step g) with 1H-indol-6-ylboronic acid provided the title product (41.8 mg, 0.088 mmol, 47%). LCMS (ES⁺) m/z 467.4 [M+H]⁺.

### Example 39

### 3-{[(3R)-1-(cyclopropylcarbonyl)-3-pyrrolidinyl]methyl}-2-(4′-fluoro-4-biphenylyl)-5,6-dimethyl-4(3H)-pyrimidinone

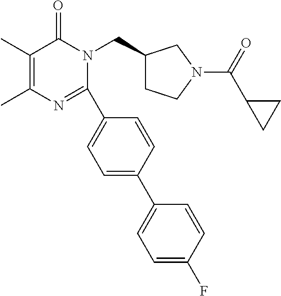 [SMILES: Cc1nc(-c2ccc(-c3ccc(F)cc3)cc2)n(C[C@@H]2CCN(C(=O)C3CC3)C2)c(=O)c1C]

Following the procedure described in Example 36 (step g) with (4-fluorophenyl)boronic acid provided the title product (21 mg, 0.046 mmol, 13.2%). LCMS (ES⁺) m/z 446.4 [M+H]⁺.

### Example 40

### 3-{[(3R)-1-(cyclopropylcarbonyl)-3-pyrrolidinyl]methyl}-2-[2-fluoro-4-(1H-indol-6-yl)phenyl]-5,6-dimethyl-4(3H)-pyrimidinone

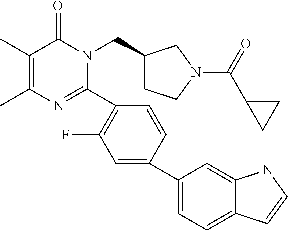 [SMILES: Cc1nc(-c2ccc(-c3ccc4cc[nH]c4c3)cc2F)n(C[C@@H]2CCN(C(=O)C3CC3)C2)c(=O)c1C]

a) 2-(4-bromo-2-fluorophenyl)-3-{[(3R)-1-(cyclopropylcarbonyl)-3-pyrrolidinyl]methyl}-5,6-dimethyl-4(3H)-pyrimidinone

Following the procedure described in Example 36 (step f) replacing 4-bromobenzamide with 4-bromo-2-fluorobenzamide provided the title product (313 mg, 0.684 mmol, 38%). LCMS (ES⁺) m/z 449.0 [M+H]⁺.

### b) 3-{[(3R)-1-(cyclopropylcarbonyl)-3-pyrrolidinyl]methyl}-2-[2-fluoro-4-(1H-indol-6-yl)phenyl]-5,6-dimethyl-4(3H)-pyrimidinone

Following the procedure described in Example 36 (step g) with 2-(4-bromo-2-fluorophenyl)-3-{[(3R)-1-(cyclopropylcarbonyl)-3-pyrrolidinyl]methyl}-5,6-dimethyl-4(3H)-pyrimidinone and 1H-indol-6-ylboronic acid provided the title compound (40 mg, 0.081 mmol, 36%). LCMS (ES⁺) m/z 485.4 [M+H]⁺.

### Example 41

### 2-[4-(1-benzofuran-5-yl)-2-fluorophenyl]-3-{[(3R)-1-(cyclopropylcarbonyl)-3-pyrrolidinyl]methyl}-5,6-dimethyl-4(3H)-pyrimidinone

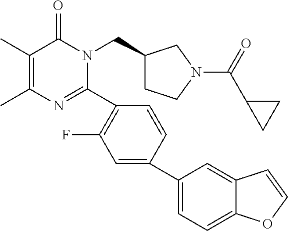 [SMILES: Cc1nc(-c2ccc(-c3ccc4occc4c3)cc2F)n(C[C@@H]2CCN(C(=O)C3CC3)C2)c(=O)c1C]

Following the procedure described in Example 36 (step g) with 2-(4-bromo-2-fluorophenyl)-3-{[(3R)-1-(cyclopropylcarbonyl)-3-pyrrolidinyl]methyl}-5,6-dimethyl-4(3H)-pyrimidinone and 5-(4,4,5,5-tetramethyl-1,3,2-dioxaborolan-2-yl)-1-benzofuran provided the title product (47 mg, 0.096 mmol, 43.2%). LCMS (ES⁺) m/z 486.4 [M+H]⁺.

### Example 42

### 3-{[(3R)-1-(cyclopropylcarbonyl)-3-pyrrolidinyl]methyl}-2-(3,4′-difluoro-4-biphenylyl)-5,6-dimethyl-4(3H)-pyrimidinone

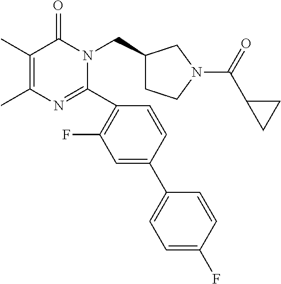 [SMILES: Cc1nc(-c2ccc(-c3ccc(F)cc3)cc2F)n(C[C@@H]2CCN(C(=O)C3CC3)C2)c(=O)c1C]

Following the procedure described in Example 36 (step g) with 2-(4-bromo-2-fluorophenyl)-3-{[(3R)-1-(cyclopropylcarbonyl)-3-pyrrolidinyl]methyl}-5,6-dimethyl-4(3H)-pyrimidinone and (4-fluorophenyl)boronic acid provided the title product (35 mg, 0.076 mmol, 47%). LCMS (ES⁺) m/z 464.3 [M+H]⁺.

### Example 43

### 3-{[(3R)-1-(cyclopropylcarbonyl)-3-pyrrolidinyl]methyl}-5-ethyl-2-[4-(1H-indol-6-yl)phenyl]-6-methyl-4(3H)-pyrimidinone

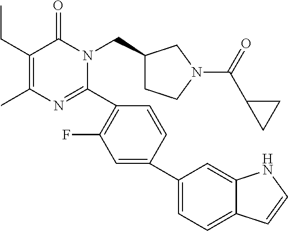 [SMILES: CCc1c(C)nc(-c2ccc(-c3ccc4cc[nH]c4c3)cc2F)n(C[C@@H]2CCN(C(=O)C3CC3)C2)c1=O]

### a) Ethyl 2-(2-ethyl-1,3-dioxolan-2-yl)propanoate

Following the procedure described in Example 36 (step a) using ethyl 2-ethyl-3-oxobutanoate provided the title compound (10 g, 49.5 mmol, 54.5% yield). LCMS (ES⁺) m/z 203.2 [M+H]⁺.

### b) 2-(2-Methyl-1,3-dioxolan-2-yl)butanoic acid

Following the procedure described in Example 36 (step b) using ethyl 2-(2-ethyl-1,3-dioxolan-2-yl)propanoate provided the title compound (4.5 g, 25.8 mmol, 56.2% yield). LCMS (ES⁺) m/z 175.2 [M+H]⁺.

### c) 2-(2-Methyl-1,3-dioxolan-2-yl)butanoyl chloride

Following the procedure described in Example 36 (step c) using 2-(2-methyl-1,3-dioxolan-2-yl)butanoic acid provided the title compound (2 g, 9.34 mmol, 90% yield). The product did not provide a peak in the LCMS.

d) N-{[(3S)-1-(cyclopropylcarbonyl)-3-pyrrolidinyl]methyl}-2-(2-methyl-1,3-dioxolan-2-yl)butanamide

Following the procedure described in Example 36 (step d) using 2-(2-methyl-1,3-dioxolan-2-yl)butanoyl chloride provided the title compound (690 mg, 2.172 mmol, 20.49% yield). LCMS (ES⁺) m/z 325.4 [M+H]⁺.

### e) N-{[(3S)-1-(cyclopropylcarbonyl)-3-pyrrolidinyl]methyl}-2-ethyl-3-oxobutanamide

Following the procedure described in Example 36 (step e) using N-{[(3S)-1-(cyclopropylcarbonyl)-3-pyrrolidinyl]methyl}-2-(2-methyl-1,3-dioxolan-2-yl)butanamide provided the title compound (500 mg, 1.78 mmol, 84% yield). LCMS (ES⁺) m/z 281.2 [M+H]⁺.

### f) 2-(4-bromophenyl)-3-{[(3R)-1-(cyclopropylcarbonyl)-3-pyrrolidinyl]methyl}-5-ethyl-6-methyl-4(3H)-pyrimidinone

Following the procedure described in Example 36 (step f) using N-{[(3S)-1-(cyclopropylcarbonyl)-3-pyrrolidinyl]methyl}-2-ethyl-3-oxobutanamide provided the title compound (310 mg, 0.656 mmol, 36.8% yield). LCMS (ES⁺) m/z 444.0 [M+H]

### g) 3-{[(3R)-1-(cyclopropylcarbonyl)-3-pyrrolidinyl]methyl}-5-ethyl-2-[4-(1H-indol-6-yl)phenyl]-6-methyl-4(3H)-pyrimidinone

2-(4-Bromophenyl)-3-{[(3R)-1-(cyclopropylcarbonyl)-3-pyrrolidinyl]methyl}-5-ethyl-6-methyl-4(3H)-pyrimidinone (70 mg, 0.158 mmol) and 1H-indol-6-ylboronic acid (27.9 mg, 0.173 mmol) were dissolved in dioxane (2 mL) in a sealable reaction vial and PdCl₂(dppf)-CH₂Cl₂ adduct (6.43 mg, 7.88 μmol) and 2N aqueous potassium carbonate (0.3 mL) were added. The vial was sealed, flushed with nitrogen and heat at 100° C. for 1 h. The reaction mixture was concentrated under reduced pressure and the residue was purified by reverse phase HPLC to provide the title compound (27.4 mg, 0.057 mmol, 36.2% yield). LCMS (ES⁺) m/z 481.3 [M+H]⁺.

### Example 44

### 2-[4-(1-benzofuran-5-yl)phenyl]-3-{[(3R)-1-(cyclopropylcarbonyl)-3-pyrrolidinyl]methyl}-5-ethyl-6-methyl-4(3H)-pyrimidinone

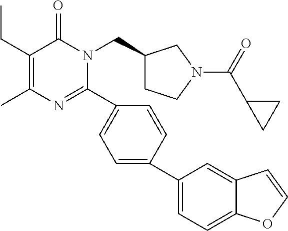 [SMILES: CCc1c(C)nc(-c2ccc(-c3ccc4occc4c3)cc2)n(C[C@@H]2CCN(C(=O)C3CC3)C2)c1=O]

Following the procedure described in Example 43 (step g) with 5-(4,4,5,5-tetramethyl-1,3,2-dioxaborolan-2-yl)-1-benzofuran provided the title product (23.6 mg, 0.048 mmol, 31%). LCMS (ES⁺) m/z 482.5 [M+H]⁺.

### Example 45

### 3-{[(3R)-1-(cyclopropylcarbonyl)-3-pyrrolidinyl]methyl}-5-ethyl-2-[4-(1H-indol-5-yl)phenyl]-6-methyl-4(3H)-pyrimidinone

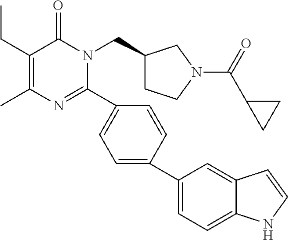 [SMILES: CCc1c(C)nc(-c2ccc(-c3ccc4[nH]ccc4c3)cc2)n(C[C@@H]2CCN(C(=O)C3CC3)C2)c1=O]

Following the procedure described in Example 43 (step g) with 5-(4,4,5,5-tetramethyl-1,3,2-dioxaborolan-2-yl)-1H-indole provided the title product (36.8 mg, 0.077 mmol, 48.6%). LCMS (ES⁺) m/z 481.6 [M+H]⁺.

### Example 46

### 3-{[(3R)-1-(cyclopropylcarbonyl)-3-pyrrolidinyl]methyl}-5-ethyl-2-(4′-fluoro-4-biphenylyl)-6-methyl-4(3H)-pyrimidinone

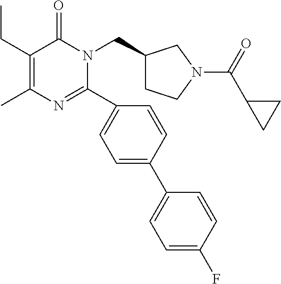 [SMILES: CCc1c(C)nc(-c2ccc(-c3ccc(F)cc3)cc2)n(C[C@@H]2CCN(C(=O)C3CC3)C2)c1=O]

Following the procedure described in Example 43 (step g) with (4-fluorophenyl)boronic acid provided the title product (37.5 mg, 0.08 mmol, 35.5%). LCMS (ES⁺) m/z 460.6 [M+H]⁺.

### Examples 47 and 48

### 2-{[(3S)-1-(cyclopropylcarbonyl)-3-pyrrolidinyl]methyl}-1-[4-(1H-indol-6-yl)phenyl]-6-methyl-4(3H)-pyrimidinone

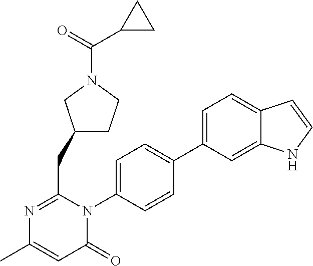 [SMILES: Cc1cc(=O)n(-c2ccc(-c3ccc4cc[nH]c4c3)cc2)c(C[C@@H]2CCN(C(=O)C3CC3)C2)n1]

2-{[(3S)-1-(cyclopropylcarbonyl)-3-pyrrolidinyl]methyl}-1-[4-(1H-indol-6-yl)phenyl]-6-methyl-4(1H)-pyrimidinone

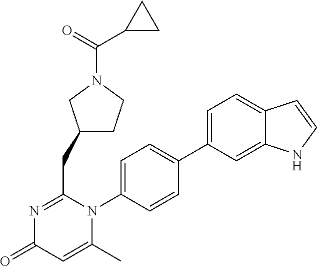 [SMILES: Cc1cc(=O)nc(C[C@@H]2CCN(C(=O)C3CC3)C2)n1-c1ccc(-c2ccc3cc[nH]c3c2)cc1]

### a) ethyl (3R)-5-oxo-1-[(1R)-1-phenylethyl]-3-pyrrolidinecarboxylate

A mixture of Itaconic acid (104 g, 800 mmol) and [(1R)-1-phenylethyl]amine (105 mL, 800 mmol) was heated at 160° C. for 2 h to give the crude acid. The acid was taken up in ethanol (200 mL) that had been acidified with conc. sulfuric acid (20 mL) and the mixture was heated under gentle reflux over for 2 days. The mixture was cooled, poured onto ice and basified with sodium hydrogen carbonate solution and extracted into ethyl acetate. The organic solution was washed with brine, dried and evaporated to give the crude ester. Gravity chromatography (silica gel 1.5 kg, 1:1 hexane:ethyl acetate) gave ethyl (3R)-5-oxo-1-[(1R)-1-phenylethyl]-3-pyrrolidinecarboxylate (83 g, 318 mmol, 79% yield). LCMS (ES⁺) m/e 261.8 [M+H]⁺.

### b) {(3R)-1-[(1R)-1-phenylethyl]-3-pyrrolidinyl}methanol

A solution of ethyl (3R)-5-oxo-1-[(1R)-1-phenylethyl]-3-pyrrolidinecarboxylate (14.8 g, 56.6 mmol) in diethyl ether (20 mL) was added dropwise to lithium aluminium hydride (1.0 molar in diethyl ether, 56.6 mL, 56.6 mmol) and was heated under reflux for 3.5 h. The mixture was cooled over an ice bath and decomposed by the careful addition of an ether-water 4:1 mixture (60 mL). The suspension was stirred at rt for 30 minutes and filtered through a Celite® pad, washed through with diethyl ether and evaporated to give {(3R)-1-[(1R)-1-phenylethyl]-3-pyrrolidinyl}methanol (11.3 g, 55.0 mmol, 97% yield) as a clear oil. LCMS (ES⁺) m/e 205.9 [M+H]⁺.

### c) (3R)-3-(chloromethyl)-1-[(15)-1-phenylethyl]pyrrolidine

A refluxing solution of {(3R)-1-[(15)-1-phenylethyl]-3-pyrrolidinyl}methanol (9 g, 43.8 mmol) in chloroform (100 mL) was treated dropwise with a solution of thionyl chloride (150 mL, 1813 mmol) in chloroform (120 mL). The mixture was heated under reflux for 2 h and evaporated. The mixture was taken up in water and the pH adjusted to 8 with 2 molar aqueous sodium hydroxide. The mixture was extracted with ethyl acetate (x3) and the combined extracts were concentrated and purified by flash chromatography (silica gel 120 g, 10-50% ethyl acetate-hexane), viewing fractions by TLC (1:1 ethyl acetate hexane), to give (3R)-3-(chloromethyl)-1-[(1S)-1-phenylethyl]pyrrolidine (8.6 g, 38.4 mmol, 88% yield). LCMS (ES⁺) m/e 225.2 [M+H]⁺.

### d) {(3S)-1-[(1S)-1-phenylethyl]-3-pyrrolidinyl}acetonitrile

A mixture of (3R)-3-(chloromethyl)-1-[(1S)-1-phenylethyl]pyrrolidine (8.5 g, 38.0 mmol), sodium cyanide (9.31 g, 190 mmol) and N-methyl-N,N-dioctyl-1-octanaminium chloride (300 mg, 0.742 mmol) in water (20 mL) was vigorously stirred at 100° C. for 48 h. The mixture was cooled and diluted with ethyl acetate. The layers were separated and the aqueous extracted with ethyl acetate (x2). The combined extracts were washed with brine, dried and evaporated. Flash chromatography (silica gel (100 g, hexane and stepwise elution to 50% ethyl acetate in hexane then 90% ethyl acetate in hexane) gave {(3S)-1-[(1S)-1-phenylethyl]-3-pyrrolidinyl}acetonitrile (4.96 g, 23.14 mmol, 60.9% yield). LCMS (ES⁺) m/e 215.0 [M+H]⁺. (3R)-3-(Chloromethyl)-1-[(1S)-1-phenylethyl]pyrrolidine (3.5 g) was also recovered.

### e) i. (3S)-3-pyrrolidinylacetonitrile

A mixture of {(3S)-1-[(1S)-1-phenylethyl]-3-pyrrolidinyl}acetonitrile (4.5 g, 21.0 mmol), ammonium formate (2.90 g, 42.0 mmol) and 10% Pd/C in methanol (50 mL) was vigorously stirred at 60° C. for several hours. The mixture was cooled and filtered through a PTFE frit, washed through with ethanol and evaporated. To the residue in ethanol (50.0 mL) was added 20% palladium hydroxide on carbon (wet) (2.95 g, 21.0 mmol) and a further portion of ammonium formate. The mixture was then heated at 60° C. for 2 hours. The mixture was filtered through a PTFE frit and evaporated to give (3S)-3-pyrrolidinylacetonitrile (2.4 g, 21.8 mmol, 104% yield) as an oil. LCMS (ES⁺) m/e 111.1 [M+H]⁺.

### ii. [(3S)-1-(cyclopropylcarbonyl)-3-pyrrolidinyl]acetonitrile

A mixture of (3S)-3-pyrrolidinylacetonitrile (1.5 g, 13.6 mmol) and Hunig's base (11.86 ml, 68.1 mmol) in chloroform (50 mL) was cooled over an ice bath and treated with a solution of cyclopropanecarbonyl chloride (1.36 ml, 14.98 mmol) in chloroform (20 mL). The ice bath was removed and the mixture was stirred for 1 h. The mixture was washed with water and brine, dried and evaporated. Flash Chromatography (silica gel, 0-10% methanol in dichloromethane) gave [(3S)-1-(cyclopropylcarbonyl)-3-pyrrolidinyl]acetonitrile (1.6 g, 8.98 mmol, 65.9% yield). LCMS (ES⁺) m/e 178.9 [M+H]⁺.

### f) N-(4-bromophenyl)-2-[(3S)-1-(cyclopropylcarbonyl)-3-pyrrolidinyl]ethanimidamide

4-(Bromophenyl)aminochloromethyl aluminium was prepared by adding a solution of 2.0 molar trimethylaluminum in toluene (16.8 mL) dropwise to a cooled suspension of 4-bromoaniline hydrochloride (7.0 g, 33.6 mmol) in toluene (50 mL) allowing the evolution of gas to subside after each addition. The mixture was stirred at room temperature for 3 h and a portion used directly in the next step.

A freshly prepared solution of (4-bromophenyl)aminochloromethyl aluminium in toluene (17.05 ml, 7.5 mmol) (prepared as shown above) was added to [(3S)-1-(cyclopropylcarbonyl)-3-pyrrolidinyl]acetonitrile (1.30 g, 7.29 mmol) in toluene and the mixture was heated under reflux for 20 h. The mixture was cooled and silica gel was added and the mixture was diluted with chloroform (100 mL). The mixture was slurried for 5 minutes, filtered through a glass sinter funnel, washed through with methanol and evaporated to provide N-(4-bromophenyl)-2-[(3S)-1-(cyclopropylcarbonyl)-3-pyrrolidinyl]ethanimidamide as a brown gum that was carried on crude without further purification. LCMS (ES⁺) m/e 351.1 [M+H]⁺.

### g) 1-(4-bromophenyl)-2-{[(3S)-1-(cyclopropylcarbonyl)-3-pyrrolidinyl]methyl}-6-methyl-4(1H)-pyrimidinone and 3-(4-bromophenyl)-2-{[(3S)-1-(cyclopropylcarbonyl)-3-pyrrolidinyl]methyl}-6-methyl-4(3H)-pyrimidinone

A mixture of ethyl 2-butynoate (0.252 mL, 2.17 mmol), N-(4-bromophenyl)-2-[(3S)-1-(cyclopropylcarbonyl)-3-pyrrolidinyl]ethanimidamide (380 mg, 1.085 mmol) and N,N-diisopropylethylamine (0.168 mL, 1.085 mmol) in ethanol was sealed in a microwave vessel and heated at 120° C. for 1 h. Flash chromatography (silica gel, 0-10% methanol in dichloromethane) gave a red-brown foam. Structure of the product was confirmed by nOe examination as an approximate 2:1 mixture of 1-(4-bromophenyl)-2-{[(3S)-1-(cyclopropylcarbonyl)-3-pyrrolidinyl]methyl}-6-methyl-4(1H)-pyrimidinone and 3-(4-bromophenyl)-2-{[(3S)-1-(cyclopropylcarbonyl)-3-pyrrolidinyl]methyl}-6-methyl-4(3H)-pyrimidinone (120 mg, 0.288 mmol, 26.6% yield). LCMS (ES⁺) m/e 417.2 [M+H]⁺.

### h) 2-{[(3S)-1-(cyclopropylcarbonyl)-3-pyrrolidinyl]methyl}-1-[4-(1H-indol-6-yl)phenyl]-6-methyl-4(1H)-pyrimidinone and 2-{[(3S)-1-(cyclopropylcarbonyl)-3-pyrrolidinyl]methyl}-3-[4-(1H-indol-6-yl)phenyl]-6-methyl-4(3H)-pyrimidinone

An approximate 2:1 mixture of 1-(4-bromophenyl)-2-{[(3S)-1-(cyclopropylcarbonyl)-3-pyrrolidinyl]methyl}-6-methyl-4(1H)-pyrimidinone and 3-(4-bromophenyl)-2-{[(3S)-1-(cyclopropylcarbonyl)-3-pyrrolidinyl]methyl}-6-methyl-4(3H)-pyrimidinone (30 mg, 0.072 mmol), 5-(4,4,5,5-tetramethyl-1,3,2-dioxaborolan-2-yl)-1H-indole (56.1 mg, 0.231 mmol), potassium carbonate (80 mg, 0.576 mmol) and tetrakis(triphenylphosphine)palladium(0) (8.33 mg, 7.21 μmol) in 1,4-dioxane (3 mL) and water (1 mL) was sealed in a pressure vessel and heated at 120° C. for 1 h over an oil bath. The mixture was partitioned between ethyl acetate and dilute brine. The aqueous was extracted with ethyl acetate and the combined extracts were washed with brine, dried, filtered and evaporated. Purification by Prep HPLC (Luna C18(2) 5u 21.2×250 mm, eluting with 65:35 300 mM aqueous ammonium formate (pH 4): acetonitrile @ 20 mL/min and detected at 254 nm) gave 2-{[(3S)-1-(cyclopropylcarbonyl)-3-pyrrolidinyl]methyl}-1-[4-(1H-indol-6-yl)phenyl]-6-methyl-4(1H)-pyrimidinone (26 mg, 0.057 mmol, 39.9% yield), MS (ES)+m/e 453.3 [M+H]⁺ and 2-{[(3S)-1-(cyclopropylcarbonyl)-3-pyrrolidinyl]methyl}-3-[4-(1H-indol-6-yl)phenyl]-6-methyl-4(3H)-pyrimidinone (12 mg, 0.027 mmol, 18.40% yield), LCMS (ES⁺) m/e 453.3 [M+H]⁺.

### Example 49

### 1-[4-(1-benzofuran-5-yl)phenyl]-2-{[(3S)-1-(cyclopropylcarbonyl)-3-pyrrolidinyl]methyl}-6-methyl-4(1H)-pyrimidinone

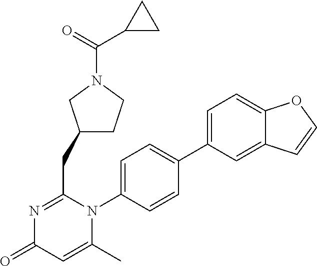 [SMILES: Cc1cc(=O)nc(C[C@@H]2CCN(C(=O)C3CC3)C2)n1-c1ccc(-c2ccc3occc3c2)cc1]

An approximate 2:1 mixture of 1-(4-bromophenyl)-2-{[(3S)-1-(cyclopropylcarbonyl)-3-pyrrolidinyl]methyl}-6-methyl-4(1H)-pyrimidinone and 3-(4-bromophenyl)-2-{[(3S)-1-(cyclopropylcarbonyl)-3-pyrrolidinyl]methyl}-6-methyl-4(3H)-pyrimidinone, 5-(4,4,5,5-tetramethyl-1,3,2-dioxaborolan-2-yl)-1-benzofuran (38.7 mg, 0.159 mmol), potassium carbonate (80 mg, 0.576 mmol) and tetrakis(triphenylphosphine)palladium(0) (8.33 mg, 7.21 μmol) in 1,4-dioxane (3 mL) and water (1 mL) was sealed in a pressure vessel and heated at 120° C. for 2 h. The mixture was partitioned between ethyl acetate and dilute brine. The aqueous layer was extracted with ethyl acetate and the combined extracts washed with brine and evaporated. Purification by Prep HPLC (Luna C18(2) 5u 21.2×250 mm eluting with 60:40 water:acetonitrile 0.1% TFA @20 mL/min and detected @ 254 nm) gavel-[4-(1-benzofuran-5-yl)phenyl]-2-{[(3S)-1-(cyclopropylcarbonyl)-3-pyrrolidinyl]methyl}-6-methyl-4(1H)-pyrimidinone (21 mg, 0.046 mmol, 32.1% yield). LCMS (ES⁺) m/e 454.1 [M+H]⁺.

### Biological Assays

### FAS Assay

### FAS Activity was Measured Through One of the Two Following Assays.

### Assay #1:

Inhibition of FAS activity can be measured based on the detection of residual NADPH substrate after the FAS assay is quenched. This assay is run as a 10 μL endpoint assay in 384-well format, where the reaction contains 20 μM malonyl-CoA, 2 μM acetyl-CoA, 30 μM NADPH and 40 nM FAS in 50 mM sodium phosphate, pH 7.0. The assay is run by sequentially dispensing 5 ul of a malonyl-CoA solution, then enzyme solution (containing the acetyl-CoA, and NADPH) into a black, low volume assay plate (Greiner 784076) pre-dispensed with 100 mL compound solutions in DMSO. The reaction is incubated at ambient temperature for 60 minutes, then quenched with 5 μL of a developing solution composed of 90 μM resazurin, 0.3 IU/ml diaphorase in 50 mM sodium phosphate, pH 7.0. The developed reaction is read on a Molecular Devices Analyst or Acquest (or equivalent) plate reader using a 530 nm excitation wavelength filter, a 580 nm emission filter, and 561 nm dichroic filter. The test compounds are prepared in neat DMSO at a concentration of 10 mM. For inhibition curves, compounds are diluted using a three fold serial dilution and tested at 11 concentrations (e.g. 25 μM-0.42 nM). Curves are analysed using ActivityBase and XLfit, and results are expressed as pIC50 values.

### Assay #2:

Inhibition of FAS can also be quantified based on the detection of the CoA products with a thio-reactive coumarin dye. This assay is run as a 10 μL endpoint assay in 384-well format, where the reaction contains 20 μM malonyl-CoA, 20 μM acetyl-CoA, 40 μM NADPH and 2 nM FAS in 50 mM sodium phosphate, pH 7.0, and 0.04% Tween-20. The assay is run by adding 5 μL enzyme solution to a black, low volume assay plate (Greiner 784076) pre-dispensed with 100 nl compound solutions in DMSO. After 30 minutes, 5 μL substrate is added, and the reaction incubated at ambient temperature for an additional 60 minutes. The reaction is then quenched with 10 μL of 6M guanidine-HCl containing 50 μM CPM (7-diethylamino-3-(4′-maleimidylphenyl)-4-methylcoumarin (CPM; thio-reactive dye), and incubated for 30 minutes. The plate is read on an Envision (PerkinElmer) or equivalent plate reader using a 380 nm excitation wavelength filter, and a 486 nm emission filter. Data fitting and compound preparations are done as described above.

### Lipogenesis Assay

Cultured primary human pre-adipocytes (Zen-Bio, Cat#ASCO62801) are plated at confluence (3×10⁴ cells/well) in 96-well plates (Costar, Cat#3598) coated with 0.2% gelatin (Sigma, Cat#G-6650) in DMEM/F12 medium (InvitroGen Cat#11330-032) supplemented with 10% heat inactivated fetal bovine serum (InvitroGen, Cat#16000-044). The following day (day 1) the cell differentiation is induced by replacing the seeding medium with the differentiation medium composed of DMEM/F12 medium supplemented with 10% heat inactivated fetal bovine serum, 200 μM 3-isobutyl-1-methylxanthine (Sigma, Cat#I-5879), 20 nM dexamethasone (Sigma, Cat#D-8893), 20 nM GW1929 (Sigma, Cat#G5668) and 20 nM insulin (InvitroGen, Cat#03-0110SA). On day 7, differentiation medium is replaced by the re-feed medium made of DMEM/F12 supplemented with 10% heat inactivated serum and 20 nM insulin. The appropriate concentration of tested compounds and controls are added into this medium at that time. On day 12, the relative amount of cellular triglyceride is estimated by using a Trinder kit (Sigma, Cat#TR0100). R^{e}-feed medium is aspirated and cells are washed with PBS (InvitroGen, Cat#14190-144) and the assay is performed according the kit manufacturer protocol. Briefly, reconstituted solutions A and B are mixed with 0,01% digitonin (Sigma, Cat#D-5628) prior to performing the assay and added onto the cells; plates are incubated at 37° C. for one hour. The absorbance is read at 540 nm. The data is first normalized using the following equation: 100*((UNK−Control 1)/(Control 2−Control 1)) where Control 1 is the Robust Mean of the 0% response control and Control 2 is the Robust Mean of the 100% response control. When multiple dilutions of compounds are tested, pXC50 are calculated from curves using the 4-parameter curve fitting with the following equation: y=(a−d)/(1+(s/c)̂1))+d and with IRLS (Iterative Re-weighted Least Squares) algorithms to weight outliers (Mosteller, F. & Tukey J. W. (1977) Data Analysis and Regression, pp 353-365, Addison-Wesley).

### Biological Data

Exemplified compounds of the present invention were tested according to the above assays and were found to be inhibitors of FAS. The IC₅₀ values ranged from about 1 nM to about 10 μM. The IC₅₀ values of the more active compounds range from about 1 nM to about 200 nM. The most active compounds are under 10 nM.

Each compound listed below was tested two or more times generally according to the assays described herein, and the average IC₅₀ values are listed in the table below.

| Example No. | IC₅₀ (nM) |
|---|---|
| Example 1 | 7.9 |
| Example 11 | 25.11 |
| Example 24 | 199.53 |
| Example 26 | 3.16 |
| Example 47 | 10 |
| Example 49 | 3162.43 |

1. A compound of Formula (I), 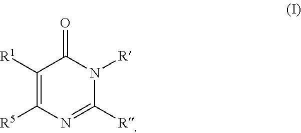
wherein, one of R′ and R″ is 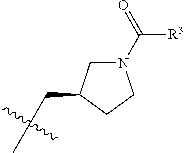
and the other of R′ and R″ is 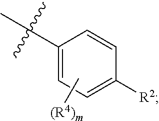
wherein
R¹ and R⁵ are each independently selected from the group consisting of: hydrogen,
C₁-C₆alkyl, —C₁-C₆alkoxy, hydroxyl, halogen,
—NR⁷R⁸, —C₁-C₆alkylNR⁷R⁸, cyano, C₄₋C₆heterocycloalkyl,
—OC₁-C₄alkyl, and —C(O)NRₐR_{b},
in which Rₐ and R_{b} are independently hydrogen, C₁-C₆alkyl, or C₃-C₇cycloalkyl, or Rₐ and R_{b} taken together with the atoms to which they are connected form a C₄-C₆heterocycloalkyl;
R⁷ is selected from the group consisting of hydrogen, C₁-C₄alkyl, C₃-C₇cycloalkyl, —C₁-C₃alkylC₃-C₇cycloalkyl, phenyl, and C₁-C₃alkylphenyl;
R⁸ is hydrogen, C₁-C₄alkyl, C₃-C₇cycloalkyl, or C₁-C₃alkylC₃-C₇cycloalkyl;
or R¹ and R⁵ taken together with the atoms to which they are connected form a 5- or 6-membered ring, which ring optionally contains one or two heteroatoms and is optionally substituted by 1 to 2 groups selected from: halogen, C₁-C₄alkoxy, and
C₁-C₄alkyl;
R² is phenyl, 5- or 6-membered heteroaryl, naphthyl, or 9- or 10-membered heterocyclyl; wherein said phenyl, 5- or 6-membered heteroaryl, naphthyl, 9- or 10-membered heterocyclyl, is optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁷R⁸, phenyl, —SO₂C₁-C₄alkyl, —SO₂NR⁷R⁸, cyano, oxo, hydroxyl, C₁-C₄alkoxy, C₃-C₇cycloalkoxy, hydroxyC₁-C₄alkyl-,C₁-C₄alkoxyC₁-C₄alkyl-, —OCF₃, —NR⁷R⁸, R⁷R⁸NC₁-C₄alkyl-, —NR⁷C(O)C₁-C₄alkyl, —NR⁷CONR⁷R⁸, —NR⁷SO₂C₁-C₄alkyl, —NR⁷SO₂NR⁷R⁸, and R⁹;
R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and NR⁷R⁸;
R³ is selected from the group consisting of C₁-C₆alkyl, —CF₃, C₃-C₇cycloalkyl, C₁-C₄alkoxy, OC₁₋₆alkyl, R⁷R⁸NC₁-C₄alkyl-, and —NR⁷R⁸; wherein said C₃-C₇cycloalkyl is optionally substituted 1 or 2 times independently by halogen or C₁-C₄alkyl;
each R⁴ is selected from the group consisting of: hydroxyl, C₁-C₆alkyl, C₁-C₆alkoxy and halogen;
m is 0 to 3; or a pharmaceutically acceptable salt thereof.

2. A compound of claim 1 represented by Formula (I)(A), 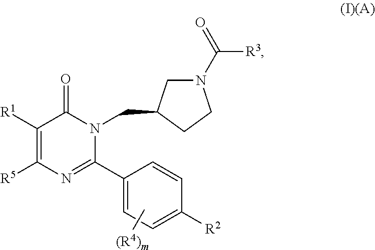
wherein
R¹ and R⁵ are each independently selected from the group consisting of: hydrogen, C₁-C₆alkyl, —C₁-C₆alkoxy, hydroxyl, halogen, —NR⁷R⁸, —C₁-C₆alkylNR⁷R⁸, cyano, C₄-C₆heterocycloalkyl, —OC₁-C₄alkyl, and —C(O)NRₐR_{b},
in which Rₐ and R_{b} are independently hydrogen, C₁-C₆alkyl, or C₃-C₇cycloalkyl, or Rₐ and R_{b} taken together with the atoms to which they are connected form a C₄-C₆heterocycloalkyl;
R⁷ is selected from the group consisting of hydrogen, C₁-C₄alkyl, C₃-C₇cycloalkyl, —C₁-C₃alkylC₃-C₇cycloalkyl, phenyl, and —C₁-C₃alkylphenyl;
R⁸ is hydrogen, C₁-C₄alkyl, C₃-C₇cycloalkyl, or —C₁-C₃alkylC₃-C₇cycloalkyl;
or R¹ and R⁵ taken together with the atoms to which they are connected form a 5- or 6-membered ring, in which the ring optionally contains one or two heteroatoms and is optionally substituted by 1 to 2 groups selected from: halogen, C₁-C₄alkoxy,
and C₁-C₄alkyl;
R² is selected from the group consisting of: phenyl, 5- or 6-membered heteroaryl, naphthyl, or 9- or 10-membered heterocyclyl; wherein said phenyl, 5- or 6-membered heteroaryl, naphthyl, 9- or 10-membered heterocyclyl, is optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl,
—CO(phenyl), —C₁-C₄(═O)OH, —C(═O)OC₁-C₄alkyl, —CONR⁷R⁸, phenyl, —SO₂C₁-C₄alkyl, —SO₂NR⁷R⁸, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁷R⁸, R⁷R⁸NC₁-C₄alkyl-, —NR⁷C(O)C₁-C₄alkyl, —NR⁷CONR⁷R⁸, —NR⁷SO₂C₁-C₄alkyl,
—NR⁷SO₂NR⁷R⁸, and R⁹;
R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁷R⁸_{;}
R₃ is selected from the group consisting of C₁-C₆alkyl, —CF₃, C₃-C₇cycloalkyl, C₁-C₄alkoxy, O C₁-C₆alkyl, R⁷R⁸NC₁-C₄alkyl-, and —NR⁷R⁸; wherein said C₃-C₇cycloalkyl is optionally substituted 1 or 2 times independently by halogen or C₁-C₄alkyl;
each R⁴ is selected from the group consisting of: hydroxyl, C₁-C₆alkyl, alkoxy and halogen;
m is 0 to 3; or a pharmaceutically acceptable salt thereof.

3. A compound of claim 1 represented by Formula (I)(B), 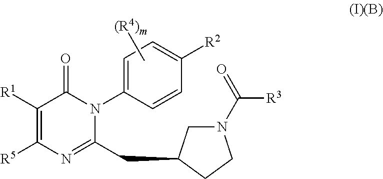
wherein
R₁ and R₅ are each independently selected from the group consisting of: hydrogen, C₁-C₆alkyl, —C₁-C₆alkoxy, hydroxyl, halogen, —NR⁷R⁸, —C₁₋₆alkylNR⁷R⁸, cyano, C₄-C₆heterocycloalkyl, —OC₁-C₄alkyl, and —C(O)NRₐR_{b},
in which Rₐ and R_{b} are independently hydrogen, C₁₋C₆alkyl, or C₃-C₇cycloalkyl, or Rₐ and R_{b} taken together with the atoms to which they are connected form a C₄-C₆heterocycloalkyl;
R⁷ is selected from the group consisting of hydrogen, C₁-C₄alkyl, C₃-C₇cycloalkyl, —C₁-C₃ alkylC₃-C₇cycloalkyl, phenyl, and C₁-C₃alkylphenyl;
R⁸ is hydrogen, C₁-C₄alkyl, C₃-C₇cycloalkyl, or C₁-C₃alkyl C₃-C₇cycloalkyl;
or R₁ and R₅ taken together with the atoms to which they are connected form a 5- or 6-membered ring, which ring optionally contains one or two heteroatoms and is optionally substituted by 1 to 2 groups selected from: halogen, C₁-C₄alkoxy, and C₁-C₄alkyl;
R² is phenyl, 5- or 6-membered heteroaryl, naphthyl, or 9- or 10-membered heterocyclyl; wherein said phenyl, 5- or 6-membered heteroaryl, naphthyl, 9- or 10-membered heterocyclyl, is optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁷R⁸, phenyl, —SO₂C₁-C₄alkyl, —SO₂NR⁷R⁸, cyano, oxo, hydroxyl, C₁-C₄alkoxy, C₃-C₇cycloalkoxy, hydroxyC₁-C₄alkyl-,C₁-C₄alkoxyC₁-C₄alkyl-, —OCF₃, —NR⁷R⁸, R⁷R⁸NC₁-C₄alkyl-, —NR⁷C(O)C₁-C₄alkyl, —NR⁷CONR⁷R⁸, —NR⁷SO₂C₁-C₄alkyl, —NR⁷SO₂NR⁷R⁸, and R⁹;
R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and NR⁷R⁸;
R³ is selected from the group consisting of C₁-C₆alkyl, —CF₃, C₃-C₇cycloalkyl, C₁-C₄alkoxy, OC₁₋₆alkyl, R⁷R⁸NC₁-C₄alkyl-, and —NR⁷R⁸; wherein said C₃-C₇cycloalkyl is optionally substituted 1 or 2 times independently by halogen or C₁-C₄alkyl
each R₄ is selected from the group consisting of: hydroxyl, C₁-C₆alkyl, C₁-C₆alkoxy
and halogen;
m is 0 to 3; or a pharmaceutically acceptable salt thereof.

4. A compound or pharmaceutically acceptable salt thereof according to claim 1, wherein R³ is cyclopropyl.

5. A compound or pharmaceutically acceptable salt thereof according to claim 1, wherein R¹ and R⁵ are each independently selected from the group consisting of: hydrogen, C₁-C₆alkyl, C₁-C₆alkoxy, hydroxyl, halogen, —NR⁷R⁸, cyano, heterocycloalkyl and —C(O)NRₐR_{b}, in which Rₐ and R_{b} are hydrogen, C₁₋C₆alkyl, C₃₋C₇cycloalkyl.

6. A compound or pharmaceutically acceptable salt thereof according to claim 1, wherein R¹ and R⁵ taken together with the atoms to which they are connected form a 5- or 6-membered ring, which ring optionally contains one or two heteroatoms atoms and is optionally substituted by 1 to 2 groups selected from: halogen, C₁-C₆alkoxy, and C₁-C₆alkyl.

7. A compound or pharmaceutically acceptable salt thereof according to claim 1, wherein m is 0.

8. A compound or pharmaceutically acceptable salt thereof according to claim 1, wherein m is 1.

9. A compound or pharmaceutically acceptable salt thereof according to claim 1, wherein R² is phenyl optionally substituted
with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-,
—NHC(O)C₁-C₄alkyl, —NHCONR⁵R⁶, —NHSO₂C₁-C₄alkyl,
—NHSO₂NR⁵R⁶, and R⁹.

10. A compound or pharmaceutically acceptable salt thereof according to claim 1, wherein R² is selected from furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl, all of which are optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —C(O)phenyl, —C₁-C₄(═O)OH, —C(═O)OC₁-C₄alkyl, —CO₂C₁-C₄alkyl, —C(O)NR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, and —NHSO₂NR⁵R⁶.

11. A compound or pharmaceutically acceptable salt thereof according to claim 1, wherein R² is naphthyl optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, —NHSO₂NR⁵R⁶, and R⁹.

12. A compound or pharmaceutically acceptable salt thereof according to claim 1, wherein R² is selected from benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzothiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzothiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl, wherein said benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzothiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzothiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl, all of which are optionally substituted with 1 to 3 substituents independently selected from halogen, C₁-C₄alkyl, —CF₃, C₃-C₇cycloalkyl, —C(O)C₁-C₄alkyl, —C(O)C₃-C₇cycloalkyl, —C(O)phenyl,
—C₁-C₄(═O)OH, —C(═O)OC₁-C₄alkyl, —C(O)NR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)NR⁵R⁶, —NR⁶SO₂C₁-C₄alkyl, —NR⁶SO₂NR⁵R⁶, and R⁹.

13. A compound or pharmaceutically acceptable salt thereof according to claim 1, wherein R² is selected from phenyl and quinolinyl.

14. A compound or pharmaceutically acceptable salt thereof according to claim 1 selected from:
5-{[(3R)-1-(cyclopropylcarbonyl)-3-pyrrolidinyl]methyl}-6-[4-(1H-indol-5-yl)phenyl]-1-methyl-1,5-dihydro-4H-pyrazolo[3,4-d]pyrimidin-4-one;
N′-[4′-(5-{[(3R)-1-(cyclopropylcarbonyl)-3-pyrrolidinyl]methyl}-1-methyl-4-oxo-4,5-dihydro-1H-pyrazolo[3,4-d]pyrimidin-6-yl)-3-biphenylyl]-N,N-dimethylsulfamide;
5-{[(3R)-1-(cyclopropylcarbonyl)-3-pyrrolidinyl]methyl}-6-[4-(1H-indol-6-yl)phenyl]-1-methyl-1,5-dihydro-4H-pyrazolo[3,4-d]pyrimidin-4-one;
6-[4-(1-benzofuran-5-yl)phenyl]-5-{[(3R)-1-(cyclopropylcarbonyl)-3-pyrrolidinyl]methyl}-1-methyl-1,5-dihydro-4H-pyrazolo[3,4-d]pyrimidin-4-one;
5-{[(3R)-1-(cyclopropylcarbonyl)-3-pyrrolidinyl]methyl}-6-[4-(1H-indazol-5-yl)phenyl]-1-methyl-1,5-dihydro-4H-pyrazolo[3,4-d]pyrimidin-4-one;
5-{[(3R)-1-(cyclopropylcarbonyl)-3-pyrrolidinyl]methyl}-1-methyl-6-[4-(6-quinolinyl)phenyl]-1,5-dihydro-4H-pyrazolo[3,4-d]pyrimidin-4-one;
5-{[(3R)-1-(cyclopropylcarbonyl)-3-pyrrolidinyl]methyl}-1-methyl-6-[4-(7-quinolinyl)phenyl]-1,5-dihydro-4H-pyrazolo[3,4-d]pyrimidin-4-one;
6-[4-(1,3-benzothiazol-5-yl)phenyl]-5-{[(3R)-1-(cyclopropylcarbonyl)-3-pyrrolidinyl]methyl}-1-methyl-1,5-dihydro-4H-pyrazolo[3,4-d]pyrimidin-4-one;
5-[4-(1-benzofuran-5-yl)phenyl]-6-{[(3R)-1-(cyclopropylcarbonyl)-3-pyrrolidinyl]methyl}-2-methyl[1,3]oxazolo[5,4-d]pyrimidin-7(6H)-one;
4′-(6-{[(3R)-1-(cyclopropylcarbonyl)-3-pyrrolidinyl]methyl}-2-methyl-7-oxo-6,7-dihydro [1,3]oxazolo[5,4-d]pyrimidin-5-yl)-4-biphenylcarbonitrile;
6-{[(3R)-1-(cyclopropylcarbonyl)-3-pyrrolidinyl]methyl}-5-[4-(1H-indazol-5-yl)phenyl]-2-methyl[1,3]oxazolo[5,4-d]pyrimidin-7(6H)-one;
5-[4-(1,3-benzothiazol-5-yl)phenyl]-6-{[(3R)-1-(cyclopropylcarbonyl)-3-pyrrolidinyl]methyl}-2-methyl[1,3]oxazolo[5,4-d]pyrimidin-7(6H)-one;
6-{[(3R)-1-(cyclopropylcarbonyl)-3-pyrrolidinyl]methyl}-5-[4-(1H-indol-5-yl)phenyl]-2-methyl[1,3]oxazolo[5,4-d]pyrimidin-7(6H)-one;
6-{[(3S)-1-(cyclopropylcarbonyl)-3-pyrrolidinyl]methyl}-5-[4-(1H-indol-6-yl)phenyl]-1-methyl-1,5-dihydro-4H-pyrazolo[3,4-d]pyrimidin-4-one;
6-{[(3S)-1-(cyclopropylcarbonyl)-3-pyrrolidinyl]methyl}-5-[4-(1H-indol-5-yl)phenyl]-1-methyl-1,5-dihydro-4H-pyrazolo[3,4-d]pyrimidin-4-one;
6-[4-(1-benzofuran-5-yl)-2-fluorophenyl]-5-{[(3R)-1-(cyclopropylcarbonyl)-3-pyrrolidinyl]methyl}-1-methyl-1,5-dihydro-4H-pyrazolo[3,4-d]pyrimidin-4-one;
5-{[(3R)-1-(cyclopropylcarbonyl)-3-pyrrolidinyl]methyl}-6-(3,4′-difluoro-4-biphenylyl)-1-methyl-1,5-dihydro-4H-pyrazolo[3,4-d]pyrimidin-4-one;
5-{[(3R)-1-(cyclopropylcarbonyl)-3-pyrrolidinyl]methyl}-6-[2-fluoro-4-(1H-indol-5-yl)phenyl]-1-methyl-1,5-dihydro-4H-pyrazolo[3,4-d]pyrimidin-4-one;
5-{[(3R)-1-(cyclopropylcarbonyl)-3-pyrrolidinyl]methyl}-6-[2-fluoro-4-(1H-indol-6-yl)phenyl]-1-methyl-1,5-dihydro-4H-pyrazolo[3,4-d]pyrimidin-4-one;
2-[4-(1-benzofuran-5-yl)phenyl]-3-{[(3R)-1-(cyclopropylcarbonyl)-3-pyrrolidinyl]methyl}-4(3H)-quinazolinone;
3-{[(3R)-1-(cyclopropylcarbonyl)-3-pyrrolidinyl]methyl}-2-[4-(1H-indol-6-yl)phenyl]-4(3H)-quinazolinone;
3-{[(3R)-1-(cyclopropylcarbonyl)-3-pyrrolidinyl]methyl}-2-[4-(1H-indol-5-yl)phenyl]-4(3H)-quinazolinone;
3-{[(3R)-1-(cyclopropylcarbonyl)-3-pyrrolidinyl]methyl}-2-[4′-(methyloxy)-4-biphenylyl]-4(3H)-quinazolinone;
2-[2-chloro-4(methoxy)-4-bipheny]-3-{[(3R)-1-(cyclopropylcarbonyl)-3-pyrrolidinyl]methyl}-4(3H)-quinazolinone;
2-[4-(1-benzofuran-5-yl)phenyl]-3-{[(3R)-1-(cyclopropylcarbonyl)-3-pyrrolidinyl]methyl}-6-methyl-4(3H)-pyrimidinone;
3-{[(3R)-1-(cyclopropylcarbonyl)-3-pyrrolidinyl]methyl}-2-[4-(1H-indol-5-yl)phenyl-6-methyl-4(3H)-pyrimidinone;
3-{[(3R)-1-(cyclopropylcarbonyl)-3-pyrrolidinyl]methyl}-2-[4-(1H-indol-6-yl)phenyl-6-methyl-4(3H)-pyrimidinone;
4-({[(3R)-1-(cyclopropylcarbonyl)-3-pyrrolidinyl]methyl}oxy)-6-methyl-2-[4′-(methyloxy)-4-biphenylyl]pyrimidine;
2-[2′-chloro-4′-(methyloxy)-4-biphenylyl]-3-{[(3R)-1-(cyclopropylcarbonyl)-3-pyrrolidinyl]methyl}-6-methyl-4(3H)-pyrimidinone;
N′-[4′-(1-{[(3R)-1-(cyclopropylcarbonyl)-3-pyrrolidinyl]methyl}-4-methyl-6-oxo-1,6-dihydro-2-pyrimidinyl)-3-biphenylyl]-N,N-dimethylsulfamide;
3-{[(3R)-1-(cyclopropylcarbonyl)-3-pyrrolidinyl]methyl}-2-(4′-fluoro-4-biphenylyl)-6-methyl-4(3H)-pyrimidinone;
2-[4-(1-benzofuran-5-yl)-2-fluorophenyl]-3-{[(3R)-1-(cyclopropylcarbonyl)-3-pyrrolidinyl]methyl}-6-methyl-4(3H)-pyrimidinone;
3-{[(3R)-1-(cyclopropylcarbonyl)-3-pyrrolidinyl]methyl}-2-[2-fluoro-4-(1H-indol-5-yl)phenyl]-6-methyl-4(3H)-pyrimidinone;
3-{[(3R)-1-(cyclopropylcarbonyl)-3-pyrrolidinyl]methyl}-2-[2-fluoro-4-(1H-indol-6-yl)phenyl]-6-methyl-4(3H)-pyrimidinone;
3-{[(3R)-1-(cyclopropylcarbonyl)-3-pyrrolidinyl]methyl}-2-(3,4′-difluoro-4-biphenylyl)-6-methyl-4(3H)-pyrimidinone;
3-{[(3R)-1-(cyclopropylcarbonyl)-3-pyrrolidinyl]methyl}-5,6-dimethyl-2-[4′-(methyloxy)-4-biphenylyl]-4(3H)-pyrimidinone;
2-[4-(1-benzofuran-5-yl)phenyl]-3-{[(3R)-1-(cyclopropylcarbonyl)-3-pyrrolidinyl]methyl}-5,6-dimethyl-4(3H)-pyrimidinone;
3-{[(3R)-1-(cyclopropylcarbonyl)-3-pyrrolidinyl]methyl}-2-[4-(1H-indol-6-yl)phenyl]-5,6-dimethyl-4(3H)-pyrimidinone;
3-{[(3R)-1-(cyclopropylcarbonyl)-3-pyrrolidinyl]methyl}-2-(4′-fluoro-4-biphenylyl)-5,6-dimethyl-4(3H)-pyrimidinone;
3-{[(3R)-1-(cyclopropylcarbonyl)-3-pyrrolidinyl]methyl}-2-[2-fluoro-4-(1H-indol-6-yl)phenyl]-5,6-dimethyl-4(3H)-pyrimidinone;
2-[4-(1-benzofuran-5-yl)-2-fluorophenyl]-3-{[(3R)-1-(cyclopropylcarbonyl)-3-pyrrolidinyl]methyl}-5,6-dimethyl-4(3H)-pyrimidinone;
3-{[(3R)-1-(cyclopropylcarbonyl)-3-pyrrolidinyl]methyl}-2-(3,4′-difluoro-4-biphenylyl)-5,6-dimethyl-4(3H)-pyrimidinone;
3-{[(3R)-1-(cyclopropylcarbonyl)-3-pyrrolidinyl]methyl}-5-ethyl-2-[4-(1H-indol-6-yl)phenyl]-6-methyl-4(3H)-pyrimidinone;
2-[4-(1-benzofuran-5-yl)phenyl]-3-{[(3R)-1-(cyclopropylcarbonyl)-3-pyrrolidinyl]methyl}-5-ethyl-6-methyl-4(3H)-pyrimidinone;
3-{[(3R)-1-(cyclopropylcarbonyl)-3-pyrrolidinyl]methyl}-5-ethyl-2-[4-(1H-indol-5-yl)phenyl]-6-methyl-4(3H)-pyrimidinone;
3-{[(3R)-1-(cyclopropylcarbonyl)-3-pyrrolidinyl]methyl}-5-ethyl-2-(4′-fluoro-4-biphenylyl)-6-methyl-4(3H)-pyrimidinone;
2-{[(3S)-1-(cyclopropylcarbonyl)-3-pyrrolidinyl]methyl}-1-[4-(1H-indol-6-yl)phenyl]-6-methyl-4(3H)-pyrimidinone;
2-{[(3S)-1-(cyclopropylcarbonyl)-3-pyrrolidinyl]methyl}-1-[4-(1H-indol-6-yl)phenyl]-6-methyl-4(1H)-pyrimidinone; and
1-[4-(1-benzofuran-5-yl)phenyl]-2-{[(3S)-1-(cyclopropylcarbonyl)-3-pyrrolidinyl]methyl}-6-methyl-4(1H)-pyrimidinone.

15. A pharmaceutical composition comprising a compound or pharmaceutically acceptable salt thereof according to claim 1 and a pharmaceutically acceptable carrier.

16. A method of treating cancer comprising administering to a human in need thereof an effective amount of a compound or pharmaceutically acceptable salt thereof or pharmaceutical composition as described in claim 1.

17. A method of claim 16 wherein the cancer is selected from the group consisting of: brain (gliomas), glioblastomas, leukemias, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, renal, ovarian, pancreatic, prostate, sarcoma, osteosarcoma, giant cell tumor of bone and thyroid.

18. A method of treating cancer in a mammal in need thereof comprising: administering to said mammal a therapeutically effective amount of
a) a compound of Formula (I), as described in claim 1 or a pharmaceutically acceptable salt thereof; and
b) at least one anti-neoplastic agent.
